(19) United States
(12) Patent Application Publication
(10) Pub. No.: US 2020/0168310 A1
(43) Pub. Date: May 28, 2020

(54) SYSTEMS FOR EVALUATING DOSAGE PARAMETERS

(71) Applicants: Jerker Westin, Hedemora (SE); Ilias Thomas, Borlange (SE); Mevludin Memedi, Orebro (SE); SENSIDOSE AB, Sollentuna (SE)

(72) Inventors: Jerker Westin, Hedemora (SE); Ilias Thomas, Borlange (SE); Mevludin Memedi, Orebro (SE); Jack Spira, Sollentuna (SE)

(21) Appl. No.: 16/617,088

(22) PCT Filed: May 30, 2018
(86) PCT No.: PCT/EP2018/064303

§ 371 Date: Nov. 26, 2019

(30) Foreign Application Priority Data
May 31, 2017 (GB) 1708624.0

(51) Int. Cl. G16H 20/10; G16H 50/50; G16H 50/70; G16H 50/30

(52) U.S. Cl. CPC G16H 20/10; G16H 50/30; G16H 50/70; G16H 50/50

(57) ABSTRACT

A system comprising a prescription-determination-module that is configured to: receive patient-model-data, wherein the patient-model-data comprises one or more dose-effect-parameters that represent how a patient is expected to react to a dose of medicament over time; apply a plurality of different dosage-parameters to the patient-model-data, in order to determine a plurality of dosage-effect-data; apply one or more selection-criteria to the plurality of dosage-effect-data in order to determine one or more of the associated dosage-parameters as selected-dosage-parameters; and provide an output-signal based on the selected-dosage-parameters.

The present disclosure relates to systems that can evaluate how different dosage parameters (such as dosage intervals or dosage amounts) can affect a patient and to methods for determining an appropriate dose of a medicament for an individual patient.

The drug therapies used to treat or otherwise control a number of chronic diseases such as, but not limited to, Parkinson's disease, epilepsy, cancer, depression, schizophrenia, attention deficit-hyperactivity disorder (ADHD) as well as other neurobehavioral disorders, diabetes, arthritis and asthma and diseases requiring anti-coagulants, anti-arrhythmics and/or analgesia often have a narrow therapeutic window and produce significant side effects when dosing is non-optimal.

The timing of doses as well as the amount of the dose is therefore critical to maintain drug levels within desired levels and it is important that administered doses are as accurate as possible to reduce the effects that can otherwise arise from over, under or imprecise dosing.

In order to administer as accurate a dose as possible EP 1 058 660 B1 describes a procedure for dosing a medicine for dispensing to a single patient from a supply of equally large units or partial doses of the medicine in the form of single tablets or pellets where each unit or partial dose contains from approximately 20 to approximately 2 weight percent of the therapeutic total dose to be administered to the patient on a single occasion.

This device and procedure allows the dispensing of highly variable doses of a medicine from a single supply of the medicine, ensuring that a clinician is able to prescribe the patient a dose of medication which is highly tailored to their particular needs.

However, clinicians typically make prescribing decisions based on an estimate of a reasonable dose for a particular patient and associated condition followed by regular qualitative observations of a patient taken over a period of weeks or possibly months. The qualitative observations often involve an assessment of the patient's appearance and physical characteristics and discussions with the patient intended to reveal their own perceptions of their response to the treatment and its side effects.

Using such methods may take a considerable time to arrive at an optimal dose and may involve a patient being under or over medicated, thus experiencing potentially treatable symptoms and/or potentially unnecessary side effects, for a relatively long period of time. This is clearly undesirable. As many conditions change over time, particularly involving increased symptoms being suffered by the patient, the same problems may be repeated many times for a single patient.

According to a first aspect of the invention, there is provided a system comprising:
a prescription-determination-module, configured to:
   receive patient-model-data, wherein the patient-model-data comprises one or more dose-effect-parameters that represent how a patient is expected to react to a dose of medicament over time;
   apply a plurality of different dosage-parameters to the patient-model-data, in order to determine a plurality of dosage-effect-data;
   apply one or more selection-criteria to the plurality of dosage-effect-data in order to determine one or more of the associated dosage-parameters as selected-dosage-parameters; and
   provide an output-signal based on the selected-dosage-parameters.

By using the patient-model-data and the selection-criteria, the output-signal can be representative of selected-dosage-parameters that are particularly beneficial for the patient.

The plurality of different dosage-parameters may comprise a plurality of different dosage-intervals between discrete doses of a medicament. The plurality of different dosage-parameters may comprise a plurality of different dosage-amounts for discrete doses of a medicament.

The selection-criterion may comprise an area-criterion, which is based on:
(i) a length of time that the dosage-effect-data spends outside an acceptable range for the dosage-effect-data, and
(ii) the extent to which the dosage-effect-data is outside the acceptable range.

Each dosage-parameter may comprise a dosage-amount and a dosage-interval. The selection-criterion may comprise an area-criterion, the application of which comprises: determining an outside-range-area based on: (i) a length of time that the dosage-effect-data spends outside an acceptable range for the dosage-effect-data, and (ii) the extent to which the dosage-effect-data is outside the acceptable range; and dividing the outside-range-area by the dosage-interval.

The medicament may comprise levodopa and/or carbidopa. The medicament may comprise a medicament selected from levodopa and/or benserazide. Additionally or alternatively, the medicament may comprise anti-epileptics, anticonvulsants, betablockers, dopamine agonists, monoamine oxidase B (MOA-B) inhibitors, treatments of dyskinesias and hyperkinetic movement disorders and treatments of attention deficit disorders (ADHD).

Anti-epileptics and anticonvulsants may be selected from acetazolamide, carbamazepine, clobazam, clonazepam, eslicarbazepine acetate, ethosuximide, gabapentin, lacosamide, lamotrigine, levetiracetam, nitrazepam, oxcarbazepine, perampanel, piracetam, phenobarbital, phenytoin, pregabalin, primidone, retigabine, rufinamide, sodium valproate, stiripentol, tiagabine, topiramate, vigabatrin, zonisamide.

Beta-blockers may be selected from atenolol, bisoprolol, carvedilol, metoprolol, nebivolol, propranolol.

Dopamine agonists may be selected from aripiprazole, phencyclidine, quinpirole, salvinorin A, apomorphine, bromocriptine, cabergoline, ciladopa, dihydrexidine, dinapsoline, doxanthrine, epicriptine, lisuride, pergolide, piribedil, pramipexole, propylnorapomorphine, quinagolide, ropinirole, rotigotine, roxindole, sumanirole, fenoldopam, amphetamine, dextroamphetamine, bupropion, lisdexamfetamine, methylphenidate or dexmethylphenidate.

MOA-B inhibitors may be selected from isocarboxazid, nialamide, phenelzine, hydracarbazine, tranylcypromine, bifemelane, moclobemide, pirlindole, toloxatone, rasagiline or selegiline

ADHD treatments may be selected from methylphenidate, dexamfetamine, lisdexamfetamine, atomoxetine or guanfacine.

The output-signal may be configured to provide a visual representation of the plurality of dosage-effect-data and the associated dosage-parameters.

The output-signal may comprise a control signal for a medicament dispenser.

The system may further comprise a medicament dispenser, that is configured to automatically dispense medicament based on the selected-dosage-parameter.

The patient-model-data may comprise one or more dose-effect-parameters that represent how a patient with Parkinson's Disease is expected to react to a dose of levodopa over time.

The prescription-determination-module may be configured to:
apply a first-dosage-parameter to the patient-model-data, in order to determine first-dosage-effect-data;
apply further-dosage-parameters, that are different to the first-dosage-parameter, to the patient-model-data in order to determine further-dosage-effect-data, until an end-criterion is satisfied.

The end-criterion may comprise one or more of: a maximum number of further-dosage-parameters have been applied; and the first- or further-dosage-effect-data satisfy a dosage-effect-threshold.

The system may further comprise:
a model-builder, configured to:
   receive a symptom-score that is associated with the patient whilst they performed an exercise, and an associated exercise-time, wherein the exercise-time is representative of a time that the patient performed the exercise,
   receive a historic-dosing-schedule that includes at least one dose-taken-time, wherein the dose-taken-time is representative of a time that the patient took a dose of medicament; and
   process the symptom-score, the associated exercise-time and the historic-dosing-schedule in order to determine the patient-model-data for the prescription-determination-module.

The system may further comprise:
a score-determination-module, configured to:
   receive sensed-motion-signals that are representative of the patient's movement whilst they are performing the exercise; and
   process the sensed-motion-signals in order to determine the symptom-score for the model-builder.

The system may further comprise: a patient-instruction-module configured to provide instructions for the patient to perform the exercises, such that the sensed-motion-signals can be acquired for the score-determination-module.

The patient-instruction-module may be further configured to provide instructions for the patient to take a dose of medicament at a predetermined dose-taken-time.

The patient-instruction-module may be further configured to:
provide the instructions for the patient to perform the exercises at exercise-times; and
determine the exercise-times with respect to the dose-taken-time.

According to a further aspect of the invention, there is provided a method, which may be a computer-implemented method, comprising:
applying a plurality of different dosage-parameters to patient-model-data, in order to determine a plurality of dosage-effect-data, wherein the patient-model-data comprises one or more dose-effect-parameters that represent how a patient is expected to react to a dose of medicament over time;
applying one or more selection-criteria to the plurality of dosage-effect-data in order to determine one or more of the associated dosage-parameters as selected-dosage-parameters; and
providing an output-signal based on the selected-dosage-parameters.

Each dosage-parameter may comprise a dosage-amount and a dosage-interval. The selection-criterion may comprise an area-criterion. Applying the area-criterion may comprise:
determining an outside-range-area based on:
   (i) a length of time that the dosage-effect-data spends outside an acceptable range for the dosage-effect-data, and
   (ii) the extent to which the dosage-effect-data is outside the acceptable range; and
dividing the outside-range-area by the dosage-interval.

The method may further comprise providing a visual representation of the plurality of dosage-effect-data and the associated dosage-parameters, based on the output-signal.

The method may further comprise automatically dispensing medicament based on the selected-dosage-parameter.

The method may further comprise:
applying a first-dosage-parameter to the patient-model-data, in order to determine first-dosage-effect-data;
applying further-dosage-parameters, that are different to the first-dosage-parameter, to the patient-model-data in order to determine further-dosage-effect-data, until an end-criterion is satisfied.

The method may further comprise:
processing a symptom-score, an associated exercise-time and a historic-dosing-schedule in order to determine the patient-model-data for the prescription-determination-module, wherein:
the symptom-score is associated with the patient whilst they performed an exercise, and the associated exercise-time is representative of a time that the patient performed the exercise, and
the historic-dosing-schedule includes at least one dose-taken-time, wherein the dose-taken-time is representative of a time that the patient took a dose of medicament.

The method may further comprise:
processing sensed-motion-signals in order to determine the symptom-score for the model-builder, wherein:
the sensed-motion-signals are representative of the patient's movement whilst they are performing the exercise.

The method may further comprise:
providing instructions for the patient to perform the exercises, such that the sensed-motion-signals can be acquired for the score-determination-module.

The method may further comprise:
providing instructions for the patient to take a dose of medicament at a predetermined dose-taken-time.

The method may further comprise:
providing the instructions for the patient to perform the exercises at exercise-times; and
determining the exercise-times with respect to the dose-taken-time.

The method may further comprise:
at least one of the dose-taken-times being representative of the patient having received a bolus dose of the medicament.

The method may further comprise:
at least one of the exercise times being before the patient receives the bolus dose.

In a further aspect, the invention provides a method of determining a personalised dosage regime for a patient for the treatment of a disease or disorder, the method comprising the steps described above. The disease or disorder may be characterised by the modulation of musculo-skeletal movement.

In some embodiments, the disease or disorder is selected from primary or idiopathic Parkinsonism, Secondary Parkinsonism, hereditary Parkinsonism, Parkinson plus syndromes, Hallevorden-Spatz Disease, progressive supranuclearophthalmoplegia, striatonigral degeneration, dystonia, spasmodic torticollis, blepharospasm, essential tremor, unspecified tremors, myoclonus, chorea, athetosis, dyskinesia, tardive dyskenisia, tic disorders, Tourette's syndrome, stereotypic movement disorder, paroxysmal nocturnal limb movement, restless leg syndrome, stiff-person syndrome, fascisculation, epilepsy, seizures or ADHD. The medicament may comprise levodopa and/or carbidopa or levodopa and/or benzerazide. In other embodiments, the treatment may include dopamine agonists such as those selected from aripiprazole, phencyclidine, quinpirole, salvinorin A, apomorphine, bromocriptine, cabergoline, ciladopa, dihydrexidine, dinapsoline, doxanthrine, epicriptine, lisuride, pergolide, piribedil, pramipexole, propylnorapomorphine, quinagolide, ropinirole, rotigotine, roxindole, sumanirole, fenoldopam, amphetamine, dextroamphetamine, bupropion, lisdexamfetamine, methylphenidate or dexmethylphenidate. In still further embodiments, the treatment may include MOA-B inhibitors such as those selected from isocarboxazid, nialamide, phenelzine, hydracarbazine, tranylcypromine, bifemelane, moclobemide, pirlindole, toloxatone, rasagiline or selegiline. In still further embodiments, the treatment may be an anti-epileptic and anticonvulsant selected from acetazolamide, carbamazepine, clobazam, clonazepam, eslicarbazepine acetate, ethosuximide, gabapentin, lacosamide, lamotrigine, levetiracetam, nitrazepam, oxcarbazepine, perampanel, piracetam, phenobarbital, phenytoin, pregabalin, primidone, retigabine, rufinamide, sodium valproate, stiripentol, tiagabine, topiramate, vigabatrin, zonisamide. In still further embodiments, the treatment may include ADHD treatments selected from methylphenidate, dexamfetamine, lisdexamfetamine, atomoxetine or guanfacine. In still further embodiments, the treatment may include beta blockers selected from atenolol, bisoprolol, carvedilol, metoprolol, nebivolol, propranolol.

In a preferred embodiment, the medicament comprises levodopa and/or carbidopa and the disease or disorder is selected from primary or idiopathic Parkinsonism, Secondary Parkinsonism, hereditary Parkinsonism, Parkinson plus syndromes.

In a further aspect, the invention provides a method of treating a disease or disorder, the method including determining a personalised dosage regime for a patient according to a method as described above.

In a further aspect, the invention provides a pharmaceutical composition comprising a medicament (e.g. comprising levodopa and/or carbidopa or levodopa and/or benserazide or others as described above) for use in a method of therapy for a disease or disorder medicament is administered in a dosage regime determined according to the method as described above.

The pharmaceutical composition is preferably provided in tablet form wherein the tablets contain between 2% and 30% (preferably between 2% and 20%) of an effective individual dose of the medicament.

There is also disclosed a system comprising one or more of:
a patient-instruction-module configured to:
   provide instructions for the patient to perform exercises; and
   optionally provide instructions for the patient to take a dose of medicament at a predetermined dose-taken-time;
a score-determination-module, configured to:
   receive sensed-motion-signals that are representative of the patient's movement whilst they are performing the exercise; and
   process the sensed-motion-signals in order to determine a symptom-score that is associated with the patient whilst they performed the exercise;
a model-builder, configured to:
   receive the symptom-score, and an associated exercise-time, wherein the exercise-time is representative of a time that the patient performed the exercise,
   receive a historic-dosing-schedule that includes at least one dose-taken-time, wherein the dose-taken-time is representative of a time that the patient took a dose of medicament; and
   process the symptom-score, the associated exercise-time and the historic-dosing-schedule in order to determine a patient-model-data, wherein the patient-model-data comprises one or more dose-effect-parameters that represent how a patient is expected to react to a dose of medicament over time; and
a prescription-determination-module, configured to:
   determine one or more selected-dosage-parameters based on the patient-model-data; and
   provide an output-signal based on the one or more selected-dosage-parameters.

There may be provided a computer program, which when run on a computer, causes the computer to configure any apparatus, including a system, module or device disclosed herein or perform any method disclosed herein. The computer program may be a software implementation, and the computer may be considered as any appropriate hardware, including a digital signal processor, a microcontroller, and an implementation in read only memory (ROM), erasable programmable read only memory (EPROM) or electronically erasable programmable read only memory (EEPROM), as non-limiting examples.

The computer program may be provided on a computer readable medium, which may be a physical computer readable medium such as a disc or a memory device, or may be embodied as a transient signal. Such a transient signal may be a network download, including an internet download.

One or more embodiments will now be described by way of example only with reference to the accompanying drawings in which:

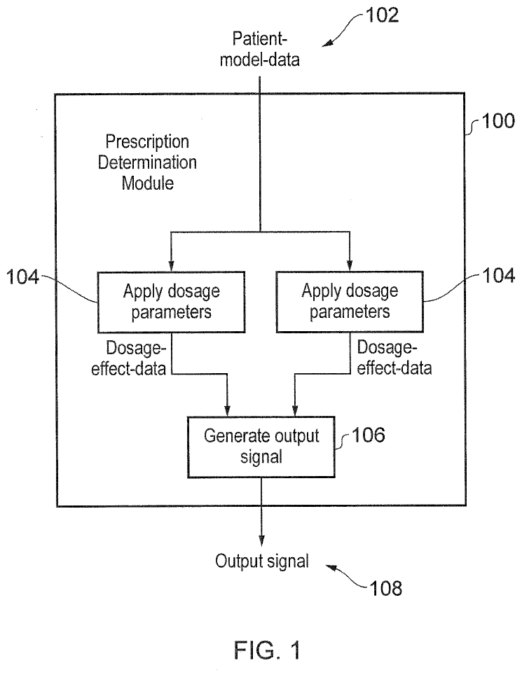

FIG. 1 shows an example of a method of processing that can be performed by a prescription-determination-module;

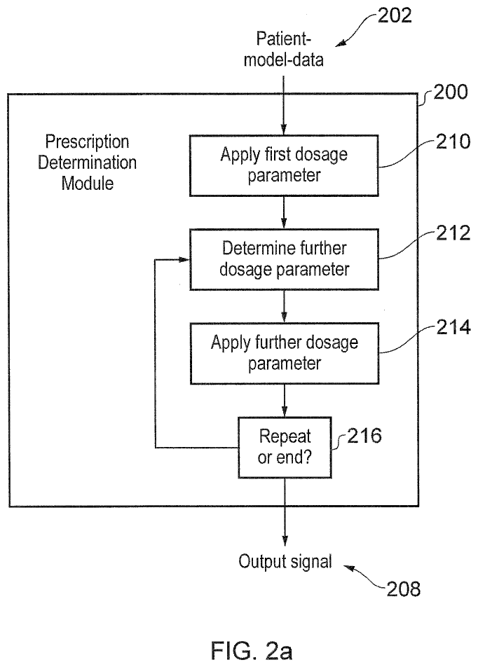

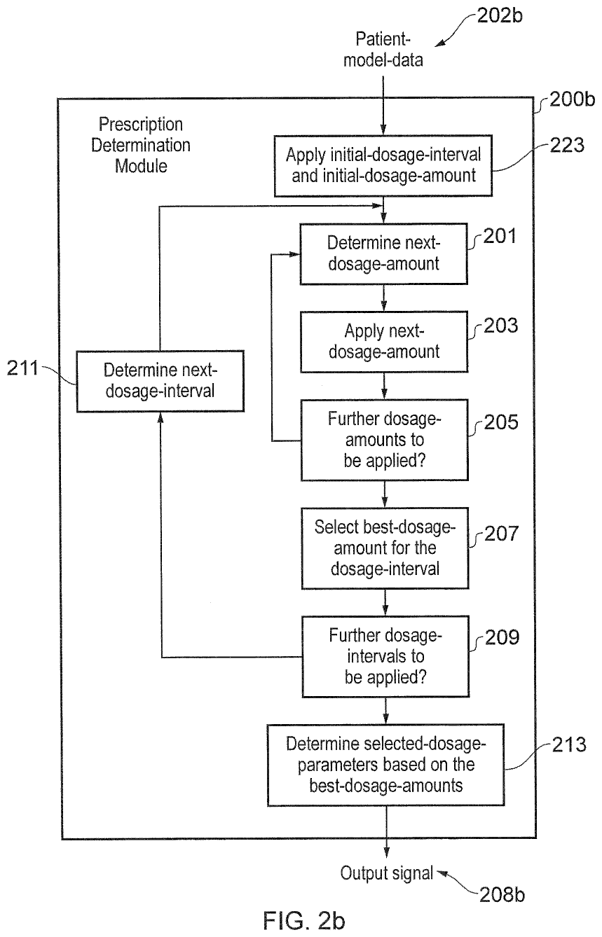

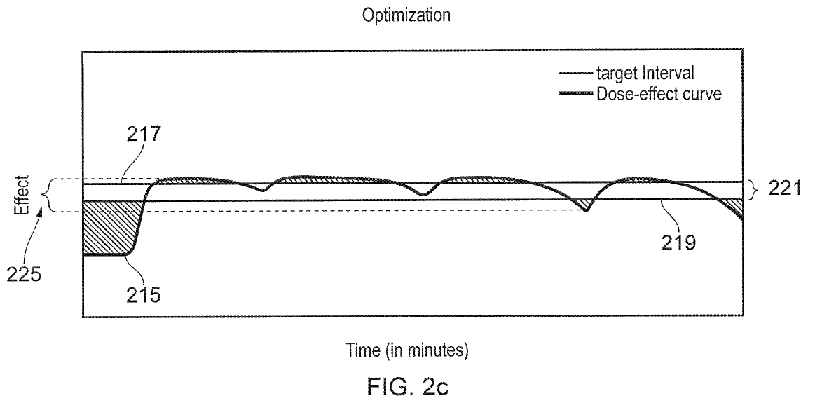

FIG. 2a shows another example of a method that can be performed by a prescription-determination-module;

FIG. 2b shows another example of a method that can be performed by a prescription-determination-module;

FIG. 2c shows graphically a plot of score on the treatment response scale (TRS) on the vertical axis, versus time on the horizontal axis;

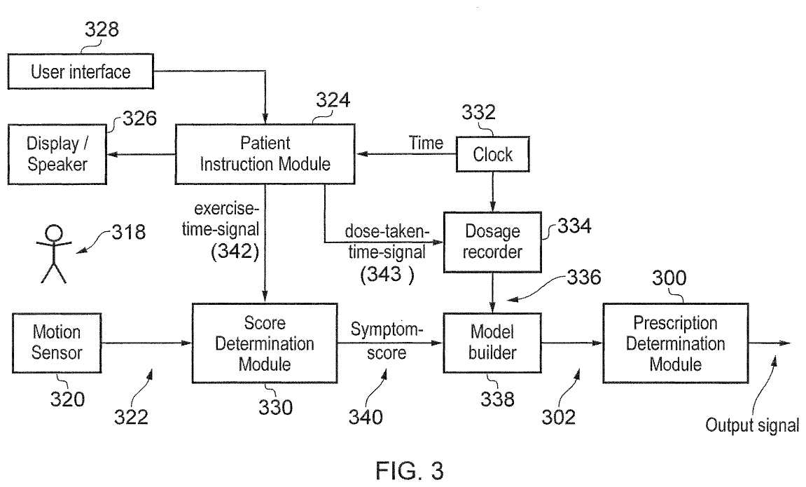

FIG. 3 shows an example of a system that includes a prescription-determination-module such as the ones illustrated in FIGS. 1 and 2;

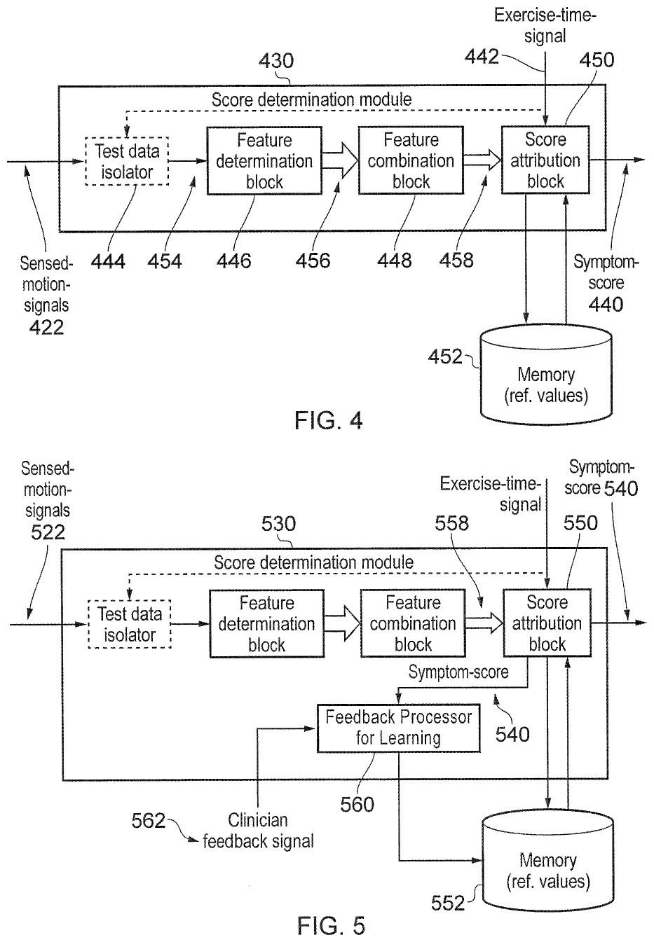

FIG. 4 illustrates an example implementation of a score-determination-module, such as the one illustrated in FIG. 3;

FIG. 5 illustrates another example implementation of a score-determination-module;

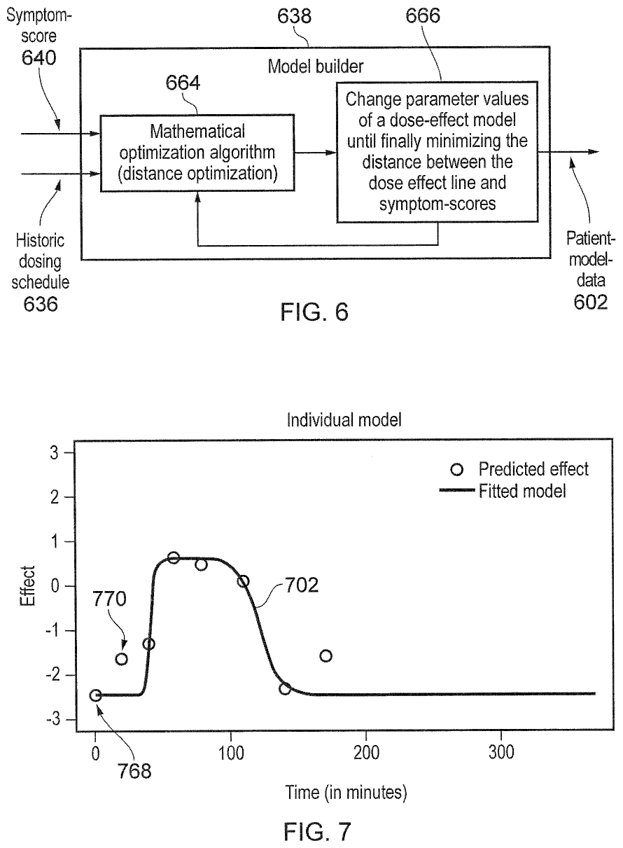

FIG. 6 shows an example implementation of a model-builder, such as the one illustrated in FIG. 3;

FIG. 7 shows graphically an example of patient-model-data;

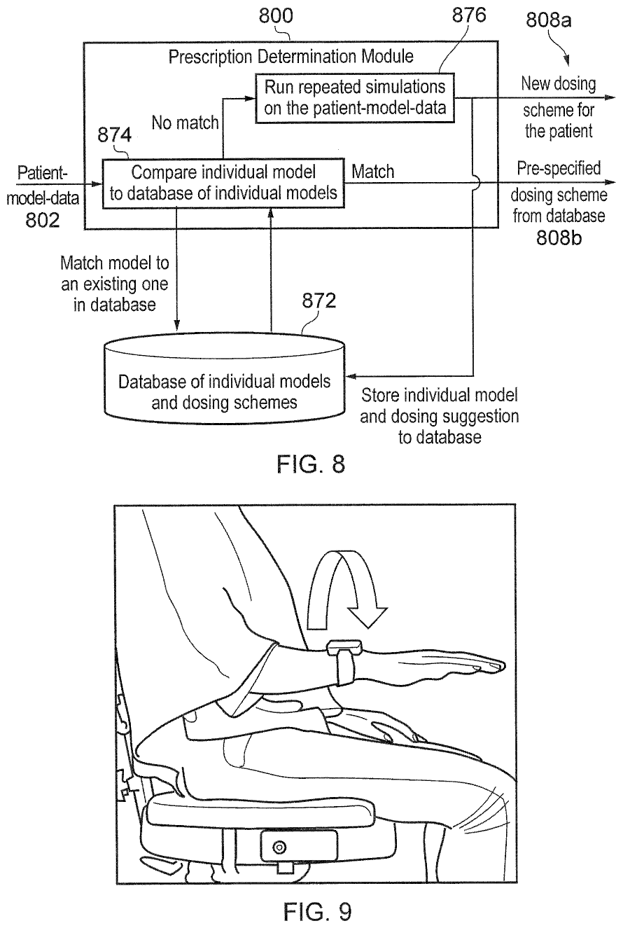

FIG. 8 shows another example of a method of processing that can be performed by a prescription-determination-module;

FIG. 9 shows a sensor and a test performed;

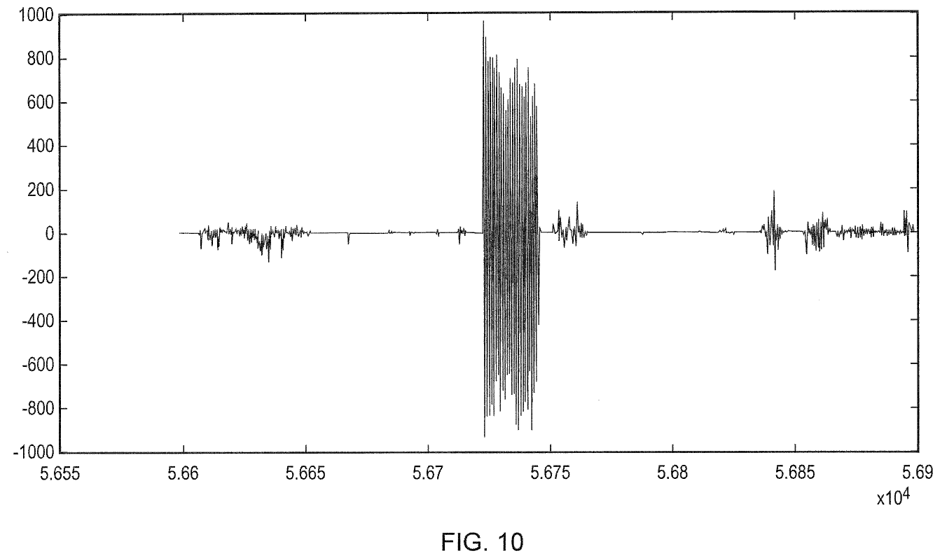

FIG. 10 shows an example visualization for a single test;

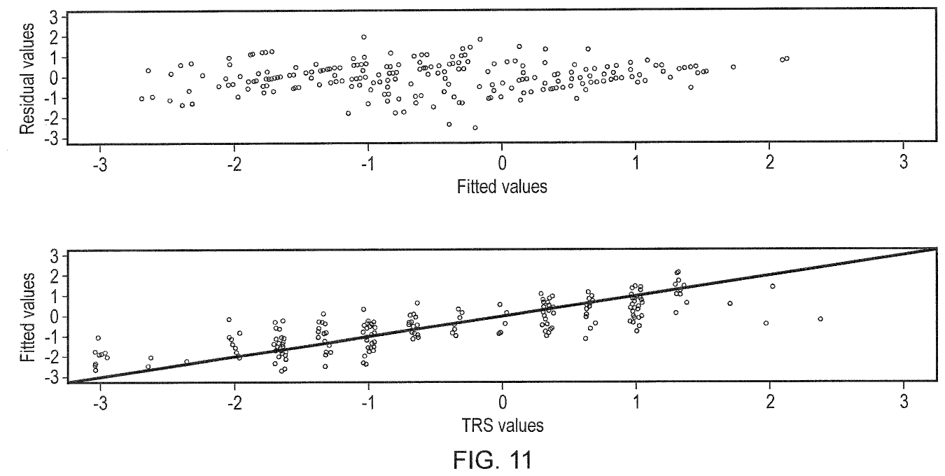

FIG. 11 shows a visual representation of the SVM (support vector machine) model performance from 10-fold cross validation;

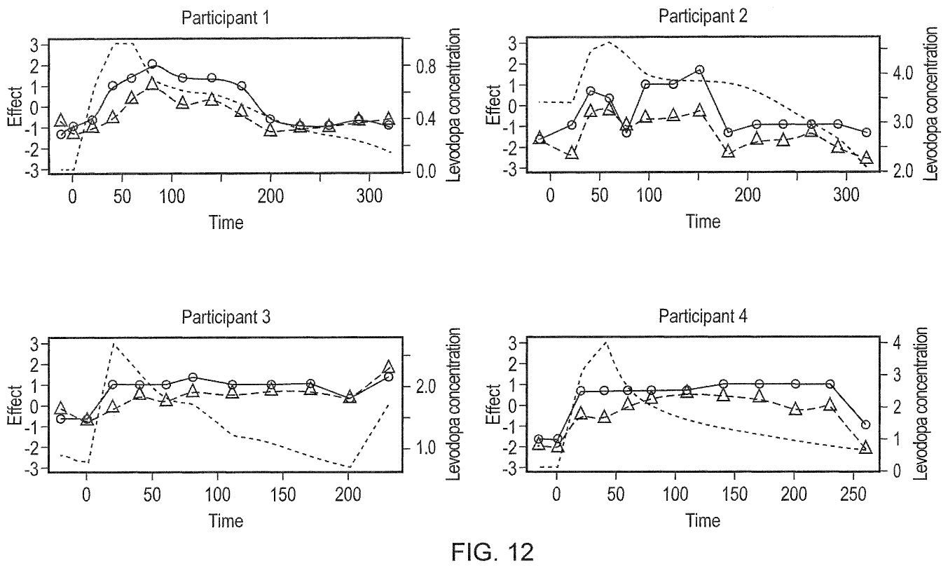

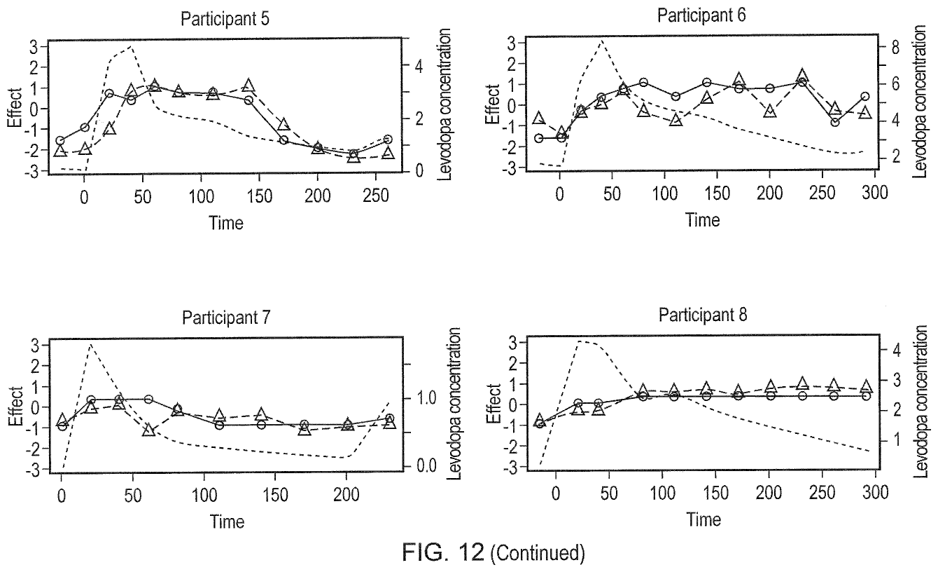

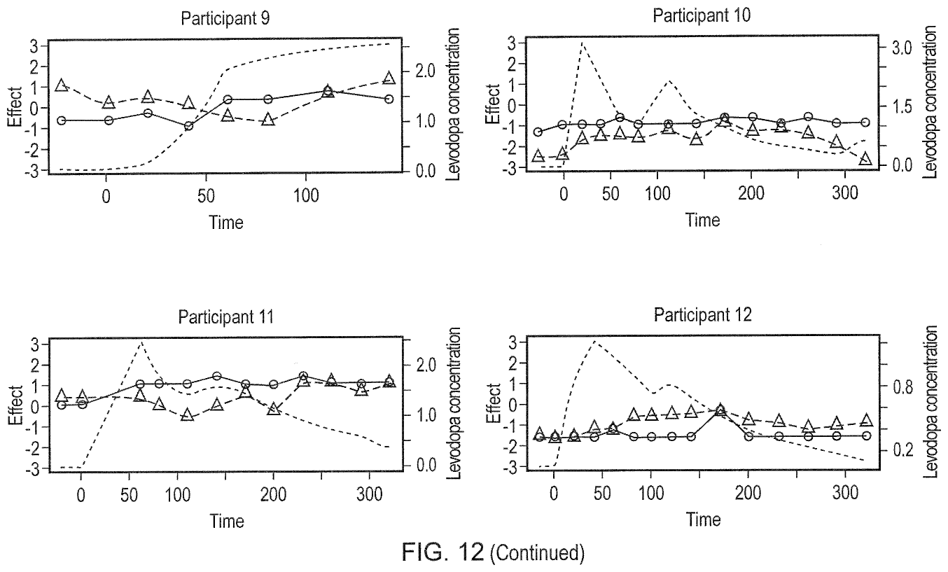

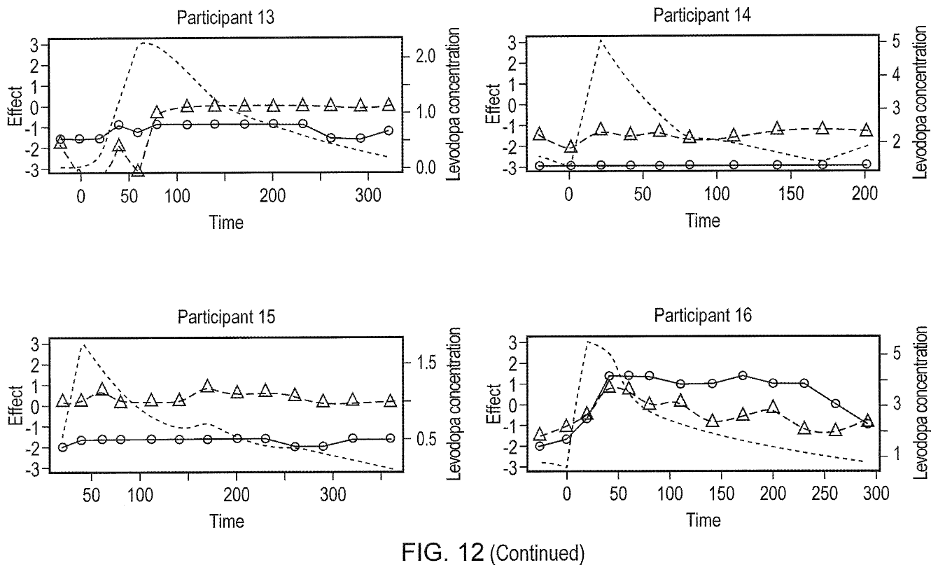

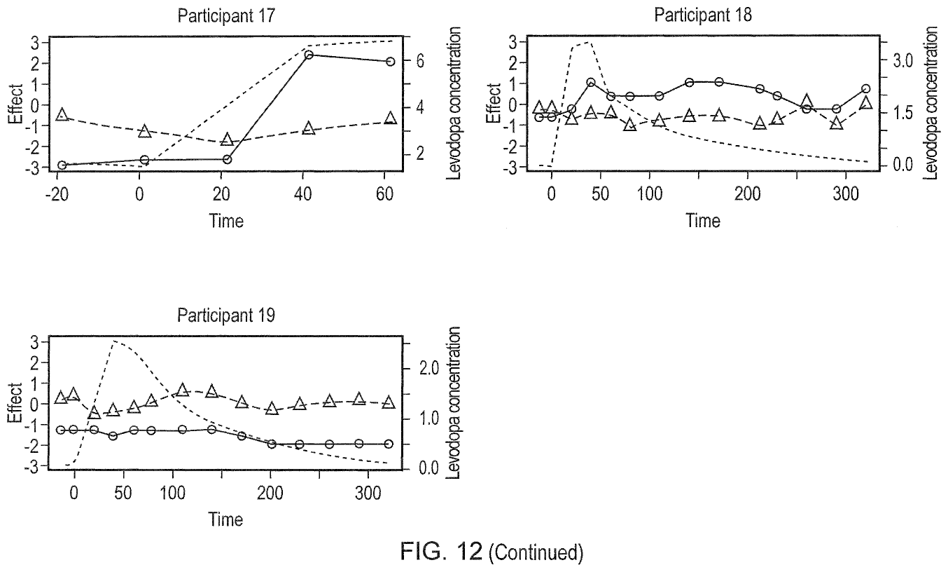

FIG. 12 shows the leave-one-individual out cross validation predictions of the SVM model;

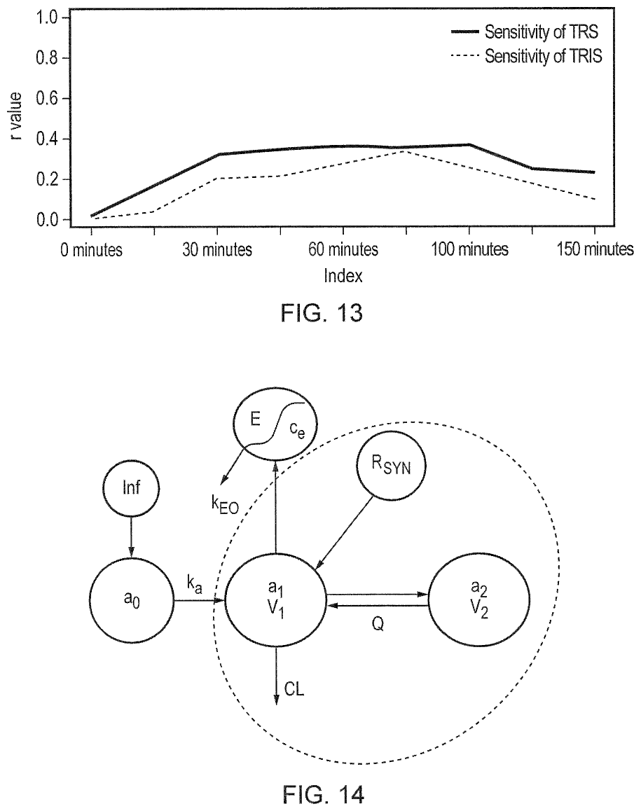

FIG. 13 shows the sensitivity of TRS (Treatment Response Scale) and TRIS (Treatment Response Index from Sensors) based on the SVM model fit;

FIG. 14 provides a graphical representation of the dynamics of the model.

One or more of the examples disclosed herein relate to a system that can evaluate how different dosage parameters (such as dosage intervals or dosage amounts) can affect a patient, such as how levodopa dosage parameters can affect a patient with Parkinson's disease. The system can also include components that can build a model of how the patient is expected to react to the different dosage parameters. In some examples, the system can build this model using signals received from a wrist sensor that is worn by the patient during predefined exercises/tests.

FIG. 1 shows an example of a method of processing that can be performed by a prescription-determination-module 100. The method can be implemented in software. The prescription-determination-module 100 receives patient-model-data 102. As will be discussed in detail below, the patient-model-data can include one or more dose-effect-parameters that represent how a patient is expected to react to medicament over time. For instance, this may include how the patient's symptoms are expected to change following a discrete dose of the medicament, or in response to continuous supply of the medicament such as can be provided by an infusion pump.

In the following description, a specific example will be described where the medicament is levodopa for treating a patient with Parkinson's disease. In such an example, the patient's symptoms can be characterised as a symptom-score using the known Treatment Response Scale (TRS), ranging from −3 (very off) to 0 (on) to +3 (very dyskinetic). Such a symptom score is an example of dosage-effect-data. However, it will be appreciated that at least some of the functionality described herein can be equally applicable to other medicaments and other conditions, which can have different scoring systems for characterising symptoms of the condition.

The prescription-determination-module 100 applies a plurality of different dosage-parameters to the patient-model-data, shown schematically as steps 104 in FIG. 1, in order to determine a plurality of dosage-effect-data. The dosage-effect-data can be representative of how the symptom-score for the patient is likely to change over time following a dose of levodopa according to the particular dosage-parameters that were applied at step 104. The different dosage-parameters can be applied by the prescription-determination-module 100 simultaneously (in parallel), or can be applied sequentially one after the other. Either way, the prescription-determination-module 100 can predict the effects on the patient's symptoms that will result from them being given different amounts of medicament, or medicament at different intervals in time, as defined by the different dosage-parameters.

The prescription-determination-module 100 can then process the dosage-effect-data at step 106 in order to provide an output-signal 108 that is based on the plurality of dosage-effect-data. Various different types of output-signal 108 can be determined by the prescription-determination-module 100.

For instance, the output-signal 108 may be representative of one or more selected-dosage-parameters, which correspond to one or more of the plurality of dosage-parameters that satisfies a selection-criterion. Examples of selection-criteria will be described below with reference to FIGS. 2a to 2c. The selected-dosage-parameters can be determined by applying an algorithm to the plurality of dosage-effect-data, or can be determined by accessing information in a database (as will be discussed below with reference to FIG. 8), or by a combination of the two. In this way, a personalised dosage regime can be determined for the patient.

Additionally, or alternatively, the output-signal 108 may be representative of:
a visual representation of the plurality of dosage-effect-data and the associated dosage-parameters. In some examples, this may be a visual representation of the dosage-effect-data that is associated with the one or more selected-dosage-parameters. In this way, the likely effects of the different dosage-parameters can be presented to a clinician so that he/she can make a prescription of the medicament for the patient with an improved understanding of how variations in the prescription are likely to affect the patient.
a control signal for a medicament dispenser, which causes the medicament dispenser to automatically dispense medicament in a way that is consistent with a selected-dosage-parameter. That is, the medicament dispenser may dispense the medicament at a dosage-time, at a dosage-interval and/or with a dosage-amount based on the selected-dosage-parameter.

FIG. 2a shows another example of a method that can be performed by a prescription-determination-module 200 The prescription-determination-module 200 can apply dosage-parameters that include one or more of: (i) a dosage-interval, which is representative of a period of time between discrete doses of a medicament; (ii) a dosage-amount, which is representative of the amount of medicament to be taken as the discrete dose; and (iii) a medicament-flow-rate, which is representative of a flow rate of a medicament that is continuously provided to the patient.

In FIG. 2a, at step 210, a first-dosage-parameter is applied to the patient-model-data 202 in order to determine first-dosage-effect-data. In this example, the first-dosage-parameter is an initial-dosage-parameter that has a value for the particular patient. For example, the initial-dosage-parameter can correspond to a dosage regime that the particular patient is currently using, or a value that has been provided by a clinician specifically for the patient (for instance, a minimum value that the clinician would be willing to prescribe). In this way, the method of FIG. 2 can start with an initial dosage parameter that is appropriate for a particular patient.

In another example, the initial-dosage-parameter can be automatically determined based on the particular patient-model-data 202 that is received. The initial-dosage-parameter can also be determined based on values received for the particular patient, as discussed above.

At step 212, a further-dosage-parameter is determined, that is different to the first-dosage-parameter. In this example, the further-dosage-parameter is determined as the next one in a predefined sequence of dosage-parameters. Such a sequence may include a sub-sequence that includes dosage-amounts that gradually increase from a minimum-dosage-amount to a maximum-dosage amount. The sequence can then include a repetition of the sub-sequence for dosage-intervals that gradually increase from a minimum-dosage-interval to a maximum-dosage interval. In this way, a plurality of different dosage-amounts can be applied for each one of a plurality of different dosage-intervals.

At step 214, the further-dosage-parameter that was determined at step 212 is applied to the patient-model-data 202 in order to determine further-dosage-effect-data.

At step 216, the method determines whether to: (i) repeat the processing of steps 212 and 214 in order to apply a new further-dosage-parameter, or (ii) end the processing and provide an output-signal 208. As discussed above, the output-signal 208 may be representative of one or more selected-dosage-parameters, which correspond to one or more of the plurality of dosage-parameters that satisfies a selection-criterion. This processing can involve determining whether or not an end-criterion is satisfied.

The end-criterion can include a maximum number of further-dosage-parameters, such that the method returns to step 212 if the maximum number have not yet been applied. In this way, the end-criterion can ensure that a complete sequence of dosage-parameters is applied.

In some examples, at step 216, the determined dosage-effect-data (including the first-dosage-effect-data and the one or more further-dosage-effect-data) can be processed in order to determine whether or not an end-criterion is satisfied. For example, the end-criterion could include a dosage-effect-threshold, such that the loop is not repeated if the first- or any further-dosage-effect-data satisfies the dosage-effect-threshold. For example, if the symptom-score is less than a predetermined threshold after a predefined period of time, and/or if the symptom-score does not change by at least a dosage-effect-iteration-threshold between iterations then an end-criterion can be considered as satisfied.

FIG. 2b shows another example of a method that can be performed by a prescription-determination-module 200b. In this example, the prescription-determination-module 200b applies dosage-parameters that have two degrees of freedom: (i) a dosage-interval, which is representative of a period of time between discrete doses of a medicament; and (ii) a dosage-amount, which is representative of the amount of medicament to be taken as the discrete dose.

As will be described below, the dosage-interval will be changed as part of an inner processing loop, and the dosage-amount will be changed as part of an outer processing loop. In this way, the system can work with repeated simulations, whereby each time a dosage-interval and dosage-amount are applied to patient-model-data 202b, a simulation is run to determine dosage-effect-data (such as a score on the TRS scale) for the specific dosage-interval and dosage-amount.

At step 223, the module determines and applies an initial-dosage-interval and an initial dosage-amount. This can be considered as running an initial simulation to determine initial-dosage-effect-data. At step 201, the module determines a next-dosage-amount. This can involve adding a predetermined dosage-amount-increment to the current dosage-amount (for the first time that step 201 is performed, the initial dosage-amount will be the current dosage-amount). Then at step 203, the next-dosage-amount that was determined at step 201 is applied to the patient-model-data 202, without changing the dosage-interval, in order to determine further-dosage-effect-data. This can be considered as performing a further simulation.

At step 205, the module checks whether or not further dosage-amounts are to be applied. This can be done by comparing the (most recently used) next-dosage-amount with a maximum-amount-threshold. If a further dosage-amount is to be applied, then the method returns to step 201 to determine the next-dosage-amount, and then apply it. This functionality can be considered as applying an inner processing loop, which applies a plurality of different dosage-amounts for a given dosage-interval.

If, at step 205, the method determines that no further dosage-amounts are to be applied, then the method moves on to step 207. At step 207, the method determines a best-dosage-amount for the specific dosage-interval that was applied. This can be performed by applying a selection-criterion to the initial-dosage-effect-data and the one or more further-dosage-effect-data. One example of a selection-criterion will now be described with reference to FIG. 2c.

FIG. 2c shows graphically a plot of dosage-effect-data (in this example a score on the treatment response scale (TRS)) on the vertical axis, versus time on the horizontal axis. A predicted dose-effect curve 215 is shown, which represents a plurality of calculated dosage-effect-data. In FIG. 2c, a period of time is shown over which four dosage-events occur—each one associated with a subsequent increase in the TRS score on the vertical axis.

Also shown in FIG. 2c is a target value 217 for the TRS score, and an acceptable-range 221 for the dose-effect curve 215. In this example, the acceptable-range 221 is bounded by a minimum-acceptable-effect-value 219 and the target value 217. In other examples, a maximum-acceptable-effect-value may also be used to define the upper boundary of the acceptable-range 221.

The “quality” of the dosage parameters can then be assessed by determining an outside-range-area, which is defined by portions of the predicted dose-effect curve 215 that are outside the acceptable-range 221. This outside-range-area is shaded in FIG. 2c. In some examples, the dosage-amount that results in the lowest value for the outside-range-area can be selected as the best-dosage-amount for that particular dosage-interval. In this way, the selection-criterion may be an area-criterion, which can be based on: (i) a length of time that the dosage-effect-data spends outside the acceptable range 221, and (ii) the extent to which the dosage-effect-data is outside the acceptable range 221.

In some examples, the method applies different or additional selection-criteria to select the best-dosage-amount at step 207 (FIG. 2b). In examples where a plurality of sub-selection-criteria are applied, the method can multiply the results of the sub-selection-criteria by weighting-factors in order to determine weighted-selection-scores, and then add the weighted-selection-scores together in order to make the final selection of the best-dosage-amount.

One example of an additional selection-criterion is a fluctuation-criterion. To apply the fluctuation-criterion, the method determines a dose-effect-range 225, which is the difference between a maximum-effect-value and a minimum-effect-value over a range-processing-period of time. In this example, an initial period, before and after a morning dose is excluded from the range-processing-period of time. This can be achieved by appropriate design of the algorithm, an example of which will be described below.

The dosage regime can include a “morning dose”, which is the first dose that a patient takes after waking up in the morning. The dosage regime also includes subsequent “daytime doses” (which may also be referred to as subsequent doses). The method/algorithm can treat the morning dose and daytime doses differently, as will be discussed below.

When the method is determining dosage parameters for “daytime doses”, it may process the TRS score for a range-processing-period of time that has a predetermined relationship with the timing of the morning dose. For example, the range-processing-period of time may be a period that starts after a predetermined-delay (such as 600 minutes) after the time that the morning dose was taken. This can be assumed to start after the effect of the morning dose has worn off.

In this way, one or more of the selection-criteria (such as the fluctuation-criterion) can be applied later in the day for determining dosage-parameters associated with daytime doses, but not applied for determining dosage-parameters associated with the morning dose. That is, different selection-criteria can be applied for determining the morning dose and the daytime doses.

In some examples, the fluctuation-criterion may be applied after, but not before, the effect (TRS score) reaches its maximum value. In this way, the drop in the TRS score may be prevented from going below a threshold value after the maximum value is reached. The initial period can thus be defined as the period up until the point when there is maximum onset of effect. Once the effect reaches the maximum value, then the range-processing-period of time can be considered to start, and the fluctuation-criterion can be applied. For instance, the algorithm can determine the maximum effect in the time period associated with the morning dose, and then apply the fluctuation-criterion for only the time period after the maximum has been reached.

The method can compare the dose-effect-range 225 with a dose-effect-range-threshold, and select the dosage-amount that results in the lowest difference between the dose-effect-range and the dose-effect-range-threshold as the best-dosage-amount. For example, if an effect of 1 is reached, and then before the next dose is taken the effect is not intended to fall below 0.5, then the dose-effect-range-threshold is 0.5. In some examples, the fluctuation-criterion may only be considered to be satisfied if the dose-effect-range is less than the dose-effect-range-threshold. In examples where the fluctuation-criterion is combined with the area-criterion, overall the selection-criteria can be considered as a combination of the effect duration, and the ability for the dose-effect curve 2015 to remain within a target range before dropping below a threshold value.

In this way, the best-dosage-amount can be selected as one that is a high enough dose to reach that target value 217, but low enough such that it does not overmedicate.

Once the best-dosage-amount has been selected for a specific dosage-interval, the method moves on to step 209 (FIG. 2b) to determine whether or not further dosage-intervals are to be applied. In a similar way to that discussed above with reference to the dosage-amount, this can be done by comparing the (most recently used) dosage-interval with a maximum-interval-threshold. If a further dosage-interval is to be applied, then the method performs an outer processing loop by moving to step 211 to determine the next-dosage-interval, and then steps 201, 203, 205 and 207 as discussed above. At step 211, the method can add a predetermined dosage-interval-increment to the current dosage-interval to determine the next-dosage-interval (for the first time that step 211 is performed, the initial dosage-interval will be the current dosage-interval). In this way, a best-dosage-amount is determined for each of the dosage-intervals that is applied.

The outer processing loop continues until there are no further dosage-intervals to be applied, as determined at step 209, in which case the method moves on to step 213. In this way, using discrete dosage-intervals, the method can incrementally change the dosage-interval from a minimum-dosage-interval to a maximum-dosage-interval, one interval at a time.

After the method determines, at step 209, that there are no further dosage-intervals to be applied, the method moves on to step 213. At step 213, the method determines selected-dosage-parameters based on the best-dosage-amounts that were determined for each of the different dosage-intervals. This can involve applying the same selection-criteria that were applied at step 207, or different selection-criteria, to determine the selected-dosage-parameters (which will be one of the best-dosage-amounts and the associated dosage-interval). In this way, the simulation results for each of the dosage-parameters (a best-dosage-amount and its associated dosage-interval) can be compared with each other, in order to evaluate which of the dosage-parameters should be selected.

For example, a modified area-criterion can be applied at step 213, which involves dividing the outside-range-area (discussed above) by the dosage-interval. This includes dividing by any value that is based on the dosage-interval, including the dosage interval raised to the power of x, where x is a positive number. In this way, the area-criterion can penalise lower dosage-intervals to account for any tendency of the area-criterion to select the lowest dosage-interval. Such a selection of the lowest dosage-interval will not always be the most appropriate choice because it can be too demanding for the patient, without necessarily providing sufficient benefits in return. It will be appreciated that any of the other selection-criteria described herein can also involve dividing a calculated value by the dosage-interval.

In one example, at step 213, the method can determine a target-exceeded-duration for each of the dosage-parameters, which is representative of the length of time that the dosage-effect-curve 215 is above the target value 217. The method can then select the dosage-parameter that has the lowest target-exceeded-duration. This can be considered as applying a target-exceeded-criterion as the selection-criterion.

Also, at step 213, the method can determine a below-target-duration for each of the dosage-parameters, which is representative of the length of time that the dosage-effect-curve 215 is below the target value 217. The method can then select the dosage-parameter that has the lowest below-target-duration. This can be considered as applying a below-target-criterion as the selection-criterion.

In addition, or instead, the method can determine a target-range-not-satisfied-duration for each of the dosage-parameters, which is representative of the length of time that the dosage-effect-curve 215 is outside the acceptable-range 221. The method can then select the dosage-parameter that has the lowest target-range-not-satisfied-duration. This can be considered as applying a target-range-satisfied-criterion as the selection-criterion.

As a further example, the method can determine a target-range-not-satisfied-count for each of the dosage-parameters, which is representative of the number or occasions that the dosage-effect-curve 215 is outside of the acceptable-range 221. The method can then select the dosage-parameter that has the lowest target-range-not-satisfied-count. This can be considered as applying a target-range-count-criterion as the selection-criterion.

It will be appreciated that any of these criteria could be applied as a selection-criterion at step 207, in addition to or instead of the criteria that are described with reference to step 207.

In some examples, an algorithm can be run at step 207 and/or step 213 to determine the best-dosage-amount and/or selected-dosage-parameters. The algorithm can be an optimization algorithm that minimizes an objective function in terms of the an outside-range-area. Optionally, the algorithm can penalize lower dosage-intervals to counter for the reduced areas that may be expected for lower dosage-intervals.

The algorithm can estimate the following parameters:
1) morning-dosage-amount, which is the amount of the first dosage that the patient will take in the morning, after waking up;
2) subsequent-dosage-amount, which is the amount of the subsequent dosages throughout a day, after the first dosage. These may be referred to as regular doses; and
3) dosage-interval, which is the time interval between doses throughout the day.

If that is the case, then the first dosage parameter can be an input to the algorithm to start the estimation process. It does not need to be different for every individual, it could be a universal starting point. In this way, the derivation can be performed using a dynamical approach (such as a minimization algorithm that is described in the next paragraph), and not using repeated simulations. For example, the first dosage parameters can be initial parameter values provided to the algorithm to start the minimization process. The algorithm can then search in the parameter space to find the parameter combinations (morning dose, subsequent doses, interval) that minimizes the function. These initial parameter values do not need to be individualized, instead they could be the same starting point for every individual (which may be referred to as a universal starting point).

In some examples, the algorithm can apply selection-criteria that look to minimize the outside-range-area around the target, and also apply a parameter that multiplies the outside-range-area by the number of doses that are scheduled per day (which is inversely related to the dosage-interval). By applying the dosage parameters based on the simulation results, and based on the individual patient-model-data, the algorithm can estimate the dosage-parameter that best minimizes the objective function. The algorithm can apply both a linear and a quadratic term, and select the term that results in the smallest error. Once the estimated objective function has been obtained in this way, the algorithm can use it to optimise individual dosage parameters.

FIG. 3 shows an example of a system that includes a prescription-determination-module 300 such as the ones illustrated in FIGS. 1 and 2. The system of FIG. 3 also shows various components that can provide the patient-model-data 302 as the input signal to the prescription-determination-module 300.

A motion sensor 320 is associated with a patient 318 with Parkinson's disease. The motion sensor 320 can provide a sensed-motion-signal 322 representative of movements of the patient 318. In this example, a wrist sensor that has accelerometers and gyroscopes can be used, although it will be appreciated that any other type of motion sensor, which may or may not be attached to the patient's body as a wearable sensor, can be used. A camera and associated image processor can also be considered as a motion sensor.

The system also includes a patient-instruction-module 324. The patient-instruction-module 324 provides instructions to the patient 318 to perform an exercise or test, such that the motion sensor 320 can provide sensed-motion-signals 322 that are representative of the patient's 318 movement whilst they are performing the exercise. The patient-instruction-module 324 can provide the instructions to the patient 318 using a display or speaker 326, for example. The exercise may include wrist rotation tests and/or gait tests, and the sensed-motion-signals 322 may be representative of the movement of the patient's 318 wrist during the test.

The patient-instruction-module 324 can provide the instructions for the patient 318 to perform the exercise at predetermined intervals over time, which may be periodic. To assist with this, the patient-instruction-module 324 can have access to a time-signal from a clock 332. The instances in time that the exercises are performed will be referred to as exercise-times, and may be defined by the start time and end time of the exercise.

In some examples, the patient-instruction-module 324 also provides instructions for the patient 318 to take a dose of medicament at predetermined times. In such an example, the patient-instruction-module 324 may request that the patient 318 provides confirmation that they have taken the dose, for example by providing user input to a user interface 328. The user interface 328 and the display/speaker 326 may be the same component—for example, a touch sensitive display. An instance in time that the patient takes a dose of medicament, optionally as confirmed by them interacting with the user interface 328, will be referred to as a dose-taken-time. Optionally, the patient-instruction-module 324 can determine the exercise-times with respect to a dose-taken-time. For example, at predetermined periods before and/or after a dose-taken-time.

In this example, the system includes a dosage-recorder 334 that maintains a historic-dosing-schedule. The historic-dosing-schedule includes at least one dose-taken-time, which can be based on a dose-taken-time-signal 343 received from the patient-instruction-module 324, and optionally also a dosage-taken-amount that is representative of the size of the dosage that was taken at that time. The historic-dosing-schedule can include information that relates to the most recent dosage and/or previous dosages. As will be discussed in more detail below, the dosage-recorder 334 can provide the historic-dosing-schedule 336 to a model-builder 338.

The system also includes a score-determination-module 330, which provides a symptom-score 340 to the model-builder 338, whereby the symptom-score 340 corresponds to an exercise that has been performed by the patient 318. In this example, the score-determination-module 330 also provides an exercise-time associated with the symptom-score as an output signal. As discussed above, for this application, which relates to patients with Parkinson's disease, the symptom-score can take a value in the range of −3 to +3.

The score-determination-module 330 receives the sensed-motion-signals 322 from the motion sensor 320. The score-determination-module 330 also receives an exercise-time-signal 342, that is representative of one or more exercise-times, from the patient-instruction-module 324. As will be discussed in detail below, the score-determination-module 330 can use the exercise-time-signal 342 to identify the portions of the sensed-motion-signals 322 that relate to an exercise, and can then process those portions of the sensed-motion-signals 322 to determine a symptom-score 340.

The model-builder 338 can then process the symptom-score 340, associated exercise-time, and the historic-dosing-schedule 336 to determine the patient-model-data 302 for the prescription-determination-module 300. Again, further details of an example implementation of the model-builder 338 are provided below.

The system of FIG. 3 can beneficially acquire more personalized information for the patient 318, in a convenient way because the patient can perform the exercises in their own home, and because a clinician does not need to be present to attribute a symptom-score. Also, the results of the processing (which can be one or more of a visual representation of a plurality of dosage-effect-data for different dosage-parameters, including one or more selected-dosage-parameters) can be delivered to a clinician remotely in a timely manner. In this way, the clinician can have access to more information than is currently feasible when they determine a prescription for the patient 318. For example, currently, a clinician may only be able to observe the symptoms of a patient with Parkinson's disease once or twice a year. The systems disclosed herein therefore means that the clinician can prescribe an improved dosage regime for the patient 318, in a quicker time than may be possible if the systems disclosed herein were not used. The system also allows for real-time monitoring of patients undergoing treatment such that new prescriptions can be determined and issued when required rather than when a patient's symptoms have improved or deteriorated to an extent to mean that they are being significantly over or under medicated.

Specific Example of Patient-Model-Data

A specific, non-limiting, example of patient-model-data will now be described.

Pharmacokinetics—Pharmacodynamics Model

The model selected for this analysis is the one proposed by Westin et al (Westin, Jerker, et al. “A pharmacokinetic-pharmacodynamic model for duodenal levodopa infusion.” Clinical neuropharmacology 34.2 (2011): 61-65). In that publication, a two compartment model is described as a system of differential equations. The effect is derived in the treatment response scale (TRS), a scale that ranges from −3 (very bradykinetic), to 0 (ON), to +3 (very dyskinetic) [8]. The PK system can be seen in equations 1-4.

$$\begin{matrix}\frac{{da}_{0}}{dt} = Inf - {k}_{a} * {a}_{0} & ( 1 ) \\ \frac{{da}_{1}}{dt} = BIO * {k}_{a} * {a}_{0} - ( \frac{Q + CL}{{v}_{1}} ) * {a}_{1} + ( \frac{Q}{{v}_{2}} ) * {a}_{2} + Rsyn & ( 2 ) \\ \frac{{da}_{2}}{dt} = ( \frac{Q}{{v}_{1}} ) * {a}_{1} - ( \frac{Q}{{v}_{2}} ) * {a}_{2} & ( 3 ) \\ \frac{{dc}_{e}}{dt} = kEO * ( \frac{{a}_{1}}{{v}_{1}} - {c}_{e} ) & ( 4 )\end{matrix}$$

Equation 5 describes the PD part of the system, where the output from equation 4 (concentration of levodopa in the effect compartment) is translated into an effect.

$$E = BASE + \frac{Emax * {c}_{e}^{γ}}{{c}_{e}^{γ} + EC {50}^{γ}} \qquad ( 5 )$$

FIG. 14 provides a graphical representation of the dynamics of the model and gives a clear overview of the medicine flow until effect is achieved. The graphical representation of FIG. 14 describes the PKPD characteristics (model parameters) of levodopa-carbidopa, from medicine infusion to an effect value.

In the below table an analytical description of the model parameters is given.

TABLE

| Parameter description of equations 1-5. | |
|---|---|
| Inf | Levodopa infusion |
| a0 | Concentration in first compartment (mg) |
| al | Concentration in second compartment (mg) |
| a2 | Concentration in third compartment (mg) |
| ka | Absorption rate |
| keo | Effect rate |
| BIO | Bioavailability |
| Q | Intercompartmental clearance (L/min) |
| V1 | Volume in first compartment |
| V2 | Volume in second compartment |
| CL | Clearance rate (L/min) |
| Rsyn | Endogenous levodopa synthesis rate (mg/min) |
| ce | Concentration in the effect compartment (mg) |
| EC50 | Concentration at 50% effect |
| gamma | Sigmoidicity factor |
| BASE | Baseline effect |
| Emax | Change from base effect |

Model Individualization Strategy

The first phase of the process is to build patient-specific models. In order to do this, the parameter values of the PKPD model in eq. 1-5 are altered in a way that they adapt to the dose-response behavior of the patients.

Since the amount of detail not sufficient to individualize all the parameters in the above table, it was decided that some of them would be fixed to population mean values known from published studies, and some would be individualized though an optimization process.

The parameters were divided into three groups.
1. The parameters that belong in the first group are the BASE parameter and the Emax parameter. These are determined on a patient-specific basis, as seen in the chart values. The BASE parameter describes the condition of the patient on a clinical rating scale, when the patient is unmedicated (the plasma concentration of the medicine substances attributed to the medicine is virtually 0). The Emax parameter expresses the maximum effect, when the patient is medicated.
2. The second group contains parameters that are fixed to population mean values, before the estimation process takes place.
3. In the third group are the parameters that do not have pre-defined fixed values but will be estimated using an optimization algorithm.

The distinction between the second and the third group resides in performing sensitivity analysis on the PKPD model. That analysis determines the parameters of interest. An analytical description on how it was performed follows:
First, get the confidence interval for each parameter estimate. This is calculated based on the SE or the interindividual/interoccational variability reported in the published studies (Westin, Jerker, et al. “A pharmacokinetic-pharmacodynamic model for duodenal levodopa infusion.” Clinical neuropharmacology 34.2 (2011): 61-65) (or the reported confidence intervals directly if any).
Afterwards, given a generic fixed amount of a one-time dose, observe the mean effect of the medicine on a specified time interval when using the PKPD model with all parameters set to population mean values.
Then, changing only one parameter value (not BASE and Emax) and keeping all other parameter estimates fixed to population mean values, calculate the mean effect again. The change on the parameters values is the both directions of their confidence interval, changing the population mean values proportionally to the length of the confidence interval. The same one-time dose is given when calculating the effect.
This process is repeated for all parameters.

The parameters that create the greater change are the ones that are placed in parameter group 3 and are estimated with a model fitting algorithm. The rest are placed to group 2 and are fixed to population mean values.

Model Fitting Algorithm

The following description relates to processing that is performed on “live” data to determine an instance of patient-model-data that relates to a patient for which observed data is received.

After determining which parameters require individualization, mathematical optimization is applied to find appropriate parameter values (parameter estimation process). In this process an objective function to be minimized is defined based on least squares minimization. The objective function connects the parameter estimation process to the observed data. The objective function is defined as the square root of the sum of the squared differences between observations and predictions based on a model parameter set.

In order to derive the effect, the optimization algorithm searches the parameters space, altering the parameter values of group 3. The best combination of parameter values are the ones that minimize the objective function. In other words, the algorithm finds the patient-specific parameters that produce the same dose-effect profile as the observed data, fitting a line thought the points observed. The altered parameter values together with the fixed parameters describe the individual model. The Nelder-Mead algorithm was used for the minimization process, a numerical method that uses a Simplex method for the optimization.

FIG. 4 illustrates an example implementation of a score-determination-module 430, such as the one illustrated in FIG. 3.

The score-determination-module 430 includes an optional test-data-isolator 444, which receives the exercise-time-signal 442 and the sensed-motion-signals 422. The output of the test-data-isolator 444 are isolated-sensed-motion-signals 454 that includes only those portions of the sensed-motion-signals 422 that correspond to instances in time that the patient was performing the known exercise. That is, it can remove portions of the sensed-motion-signals 422 that do not correspond to an exercise. The test-data-isolator 444 can be particularly useful in examples where the patient wears a motion sensor for periods of time before or after an exercise, so that the score-determination-module 430 can attribute a symptom-score based only on the patient's motion during the exercises.

The sensed-motion-signals 422 can be representative of any aspect of motion of the patient. Specific examples of such sensed-motion-signals 422 are described below. In some instances, the sensed-motion-signals 422 can be acquired by a gyroscope and/or an accelerometer and/or an image processor (that processes image-data/video-data received from a camera), and can be representative of one or more of:
displacement in a specific dimension, such as one or more dimensions in a Cartesian coordinate system;
speed in a specific dimension;
acceleration in a specific dimension; and
rotation about a specific dimension, including angle, speed and acceleration of rotation.

It will be appreciated that for other disorders, the sensed-motion-signals 422 can be representative of different aspects/characterisations of patient motion.

In this example, the score-determination-module 430 includes a feature-determination-block 446 that processes the isolated-sensed-motion-signals 454 (or sensed-motion-signals 422 if isolation is not required) in order to determine a plurality of feature-values 456. The feature-values 456 can be representative of any characteristic of one or more of the isolated-sensed-motion-signals 454. Specific examples of such features are described with reference to the detailed description below. In some instances, the features can be one or more of:
a statistical property of one or more of the sensed-motion-signals 422 over the duration of the entire, or part of the, exercise; such as a maximum value, a minimum value, an average value, a mean value, skewness, approximate entropy (which can be representative of an amount of irregularity in movements) or a standard deviation;
a mathematical transform of one or more of the sensed-motion-signals 422, such as a discrete wavelet transform.

The score-determination-module 430 also includes a feature-combination-block 448 in this example. The feature-combination-block 448 combines one or more of the feature-values 456 that are generated by the feature-determination-block 446 in order to determine one or more “combined values” or “reduced values”, which in this example are principal-component-values 458. The feature-combination-block 448 can apply a predefined algorithm to the feature-values 456 in order to determine the principal-component-values 458.

A score-attribution-block 450 receives the principal-component-values 458 from the feature-combination-block 448. In this example, the score-attribution-block 450 interrogates a database or look-up table stored on a memory 452 to retrieve a symptom-score 440 that corresponds to the received principal-component-values 458. The symptom-score 440 is then provided as an output signal of the score-determination-module 430. In some examples, the exercise-time 442 (which is received as an input signal) can be provided as an output signal along with the associated symptom-score 440. In other examples, the score-attribution-block 450 can apply an algorithm to the received principal-component-values 458 to determine the symptom-score 440.

FIG. 5 illustrates another example implementation of a score-determination-module 530, that is similar to the one illustrated in FIG. 4. Features of FIG. 5 that have already been described with reference to FIG. 4 will not necessarily be described again here.

The score-determination-module 530 includes a feedback-processor 560, which receives: (i) the symptom-score 540 from the score-attribution-block 550, and (ii) a clinician-feedback-signal 562, which is representative of a symptom-score that has been attributed to the patient's performance of the exercise by a clinician. In this way, the feedback-processor 560 can compare the automatically generated symptom-score 540 with the clinician-feedback-signal 562, and can then update one or more values in the memory 552 such that the automatically generated symptom-score 540 is closer to the score that has been attributed by the clinician. In examples where the score-attribution-block 550 applies an algorithm to the received principal-component-values 558, the feedback-processor 560 can update one or more coefficients of the algorithm based on the comparison.

FIG. 6 shows an example implementation of a model-builder 638, such as the one illustrated in FIG. 3.

The model-builder 638 receives one or more symptom-scores 640, and their associated exercise-times, as well as the historic-dosage-schedule 636. At step 664, the model-builder 638 then selects an initial set of dose-effect-parameters for the patient-model-data and performs a mathematical optimization algorithm (which may be a distance optimization algorithm in some examples) for the received symptom-scores 640, associated exercise-times, and historic-dosage-schedule 636. The output of step 664 can be an optimization-score that is representative of how good a fit the patient-model-data is for the received signals.

Then at step 666, the model-builder 638 changes one or more of the dose-effect-parameters, and returns to step 664 to calculate a new optimization-score for the changed dose-effect-parameters. The model-builder 638 can continue around the loop of steps 664 and 666 until the optimization-score reaches a threshold-value or a minimum value, at which point the model-builder 638 outputs the patient-model-data 602 that includes the dose-effect-parameters that are associated with the acceptable/minimum optimization-score.

FIG. 7 shows graphically an example of patient-model-data 702 that has been determined by the model-builder for received symptom-scores, their associated exercise-times, and a historic-dosage-schedule. In FIG. 7, time is shown on the horizontal axis, and symptom-score (which can also be considered as dosage effect) is shown on the vertical axis.

The first data-point 768 on the time axis represents the symptom-score, as determined by a score-determination-module, for an exercise that was performed before the dose administration. This can be considered as determining a baseline. The second data-point 770 represents the symptom-score at the time of the dose administration. The following data-points represent the symptom-scores associated with exercises performed after the dose administration. It will be appreciated that the position of the data-points on the horizontal axis are defined by the exercise-times that are associated with the received symptom-scores, and also a dose-taken-time that is represented in the historic-dosage-schedule.

Data representative of the patient-model-data 702, that is identified by the model-builder as the best fit for the data-points, is shown as line 702 in FIG. 7.

FIG. 8 shows another example method of processing that can be performed by a prescription-determination-module 800. In this example, the method determines the output signal 808a, 808b by both applying an algorithm 876 and using a database 872 of reference values.

The prescription-determination-module 800 receives patient-model-data 802, and at step 874 compares the received patient-model-data 802 with a database 872 of model-data that is stored in memory. If the prescription-determination-module 800 determines that one of the models stored in the database 872 is a close enough match, then the prescription-determination-module 800 retrieves an associated dosage-parameter from the database 872, and provides an output signal 808b that is representative of the associated dosage-parameter. A match can be determined by calculating a distance matrix associated with the received patient-model-data 802 and each of the models stored in the database 872. If the lowest value output of the distance matrix is below a certain threshold, then the prescription-determination-module 800 identifies a match. In this way, the dosage-parameter that was retrieved from the database 872 for the matching model-data can be output as a selected-dosage-parameter.

If, at step 874, the prescription-determination-module 800 does not determine a match, then the processing moves on to step 876, where simulations are run on the patient-model data for different dosage-parameters in order to select appropriate dosage-parameters for providing as an output signal 808a. That is, the prescription-determination-module 800 either: (i) provides an output signal 808b, which corresponds to pre-calculated dosage-parameters that have been retrieved from a database 872 stored in memory; or (ii) if appropriate pre-calculated dosage-parameters are not available, it provides an output signal 808a that has been calculated for the received patient-model-data 802 through simulation. Advantageously, by initially making reference to the database 872 of known models and associated dosage-parameters, the processing that is required by the prescription-determination-module 800 to provide an output signal 808a, 808b can be efficient in terms of processing time and processing resources.

In this example, when the prescription-determination-module 800 determines new dosage-parameters, because there was no match with the information stored in the database 872, it can then store the selected-dosage-parameters (determined at step 876) and the associated received patient-model-data 802 in the database 872, so that it is available as reference data for future calculations.

### DETAILED EXAMPLE

It will be appreciated that one or more of the specific processing features that is described below with reference to the detailed example can be implemented in one or more of the more general systems that are described above, without necessarily requiring all of the specific processing features that are described below.

Signal processing can be applied to extract features from the sensor data, particularly when the sensor is equipped with gyroscope and accelerometer, with the most common techniques involving the extraction of the frequency domain and the time-domain features of the signals (Mancini et al. 2012, Tsipouras at al. 2012). Calculation of features concerning the Approximate Entropy (ApEn) has also been applied in PD (Vaillancourt et al. 2000) and other neurological diseases (Abásolo et al. 2005, Guo et al. 2010). ApEn can be used to measure irregularity in movements (Pincus et al. 1991).

The features extracted can be processed with statistical methods and machine learning algorithms, both for supervised and unsupervised learning. Such methods have been used for regression and for classification purposes. For example, logistic regression has been used to predict UPDRS scores from digital voice recordings (Stamatakis et al. 2013). Support vector machines have been used in accelerometer data, to monitor motor fluctuations in PD (Patel et al. 2009). Also in accelerometer data, artificial neural networks have been successfully used to detect gait disturbances (Jane et al 2016). Machine learning was also used to identify PD states from waist worn inertial sensors (Lopane et al. 2015) (off-on-dyskinesia).

Commercial products that use machine learning algorithms are available, most notably the Parkinson's KinetiGraph™ (Griffiths et al. 2012) that produces scores for bradykinesia, tremor, dyskinesia and motor fluctuations and the Kinesia™ systems (Giuffrida et al. 2009) that produce severity scores for PD, more specifically, for tremor, bradykinesia, dyskinesia and mobility. However, there is a lack of systems that can combine scores for bradykinesia and dyskinesia in PD in the same scale, useful for personalizing dosing schedules. By doing so, a holistic way to understand medication effects could be developed, even if there is often an overlap between the phenomena of bradykinesia and dyskinesia. For that reason, in the present study the TRS scale was used (Nyholm et al. 2005).

A Treatment Response Index from Sensors (TRIS) was constructed that can map the features from the sensors to a continuous treatment response scale, from “off” to “on” to dyskinesia. The feasibility of using wrist worn motion sensors for that purpose, performing hand rotation tests, was investigated. The TRIS should have good clinimetric properties including: convergent validity, test-retest reliability and sensitivity to treatment. A TRIS was designed such that simple motor tests can be used as a way to individualize dosing routines.

2. Methods

2.1 Data Collection

2.1.1. Dataset

Nineteen participants with PD, experiencing wearing-off and/or dyskinesia, were recruited in a single center open label clinical trial. The PwP were matched with healthy subjects, and the total number of healthy individuals recruited was 22.

The trial consisted of a single levodopa dose challenge for the participants with PD. After pausing all PD medication for 12 h they were asked to perform a standardized motor task before a single levodopa-carbidopa morning dose (150% of normal dose) and at specific time intervals after the dose was given. Up to 15 samples per PwP were collected, one 20 minutes prior to dosing, one at time of dose administration (0 minutes) and then at 15, 30, 45, 60, 80, 100, 120, 150, 180, 210, 240, 300 and 360 minutes after dose administration. At each test occasion a blood sample was collected and the levodopa concentration in the blood plasma was later analyzed (Senek et al. 2016). A higher morning dose than the participants normal dose was used to increase the chance of observing dyskinesia.

The healthy controls were asked to perform the same tasks, 8 times each, at time point 0 (first test) and then at 20, 40, 60, 80, 110, 140, and 170 minutes after the first test, without receiving any PD related medication. The total number of observations was 229 for the 19 PD participants and 165 for the 22 healthy controls.

The motor task the individuals were asked to perform was a 20-second wrist pronation-supination task while wearing sensors on both wrists (the tasks were performed consecutively, right hand first). The subjects were seated on a chair without armrests while performing the tests. The Shimmer3 sensor was used for the experiment, a sensor consisting of a three dimensional accelerometer and gyroscope (102.4 Hz sampling rate, wide accelerometer range +/−16 g, gyroscope range +/−2000 dps).

FIG. 9 shows the sensor and the test performed. The subject wearing a wrist motion sensor while performing rapid alternating movements of hands. The subjects performed the tests first with the right hand and then with the left hand and each test lasted for 20 seconds.

For the sensor the X axis represented left and right, the Y axis back and forth and the Z axis vertical movement, all in relation to the subject's body position. Each test occasion was video recorded and a timestamp was embedded in each file name, corresponding to the time the test was performed.

2.1.2 Clinical Assessment of Motor Function

The video sequences were presented in a randomized order to the three movement disorder specialists, so that the ratings were blinded with respect to time from dose administration (Nyholm et al. 2005). The specialists assigned a score (from 0 to 4) to five UPDRS items: finger tapping (item 23); rapid alternating movements of hands (item 25); arising from chair (item 27); gait (item 29) and bradykinesia (item 31). Dyskinesia was also rated at the same range.

Clinical scores were also given in the Treatment Response Scale (TRS) that ranges from −3=‘Very Off’ to 0=‘On’ to +3=‘Very dyskinetic’. The clinical assessments were based on the overall motor function of the PwP and highest weight was given on the gait. The overall score for all scales at each time point was defined as the mean of the three specialists' assessments.

2.2 Data Processing and Analysis

2.2.1 Segment Identifying from Sensor Recordings

The sensors recorded continuously from the moment they were activated until the end of all the test occasions, about 6 hours of recording time for each sensor. In order to extract the segments of interest (the segments where the participants were doing the hand rotation), for each patient the timestamps corresponding to each video occasion were processed. Afterwards, the time of the recording corresponding to the timestamp of each video were extracted. Through the readings of the rotation on the Y axis, the areas of interest were able to be identified, as the movement is clearly represented in those readings. An example of the visualization for a single test can be seen in FIG. 10, where the X-axis represents time in seconds and the Y axis the sensor reading (gyroscope reading of rotation). In FIG. 10, the moment of starting and stopping the rotation is clear. The segment extracted was from 2 seconds before the movement started until 2 seconds after the movement stopped.

2.2.2 Feature Extraction

The 3D accelerometer and gyroscope measurements during rapid alternating movements of hands were processed with time series analysis methods and several spatiotemporal features were calculated and used in subsequent analysis. For each hand, 88 features were calculated.

For calculating the features, the following eight signals were used: Xacc, Yacc, Zacc, Macc, Xrot, Yrot, Zrot and Mrot. Macc and Mrot are the magnitudes of acceleration and rotation, respectively. The magnitude was calculated as the square root of the sum of the squares of the three individual axes.

The first three statistical moments (mean, standard deviation and skewness) were calculated for the eight signals to quantify their level, variation and asymmetry. These calculations resulted in 24 (3 moments multiplied by 8 signals) features. To quantify any fatigue during the hand rotation test trial, the signals were divided into two parts. The first part consisted of data points collected between 0 and 10 s and the second part consisted of data points between 10 and 20 s. The absolute mean differences between the first and second parts of the signals were calculated, which in turn resulted in 8 more features.

Irregularities in motions during the hand rotation tests were quantified by applying ApEn on the eight signals. The ApEn is a statistical method that quantifies the amount of irregularity (or randomness) in a signal. It requires setting two user-specified parameters: the length of a window (m) that is shifted throughout the signal to compare data points and the measure of similarity (r), each of which must remain fixed during calculations. In another example, m was set to 2 and r to 0.2 (20% of the signal's standard deviation), as suggested by Pincus et al. (1996). Applying ApEn in the eight signals resulted in 8 more features.

Three-level Discrete Wavelet Transform (DWT) was applied on the signals and mean and standard deviations were calculated in the frequency components. Since the sampling rate of the sensors was 102.4 Hz the maximum frequency component that could be detected in our signals was 51.2 Hz. The first level DWT resulted in low-frequency components in the frequency range 0-25.6 Hz and high frequency components in the range 25.6-51.2 Hz. In the second level, the low frequency components of the first level were further decomposed into low (0-12.8 Hz) and high (12.8-25.6 Hz) frequencies. Finally, the third level decomposed the low frequency components of the second level into low (0-6.4 Hz) and high (6.4-12.8 Hz) frequencies. Mean and standard deviation were then calculated from first level high frequencies, second level high frequencies and a combined signal, which consisted of the following components in the following order: third level low frequency components, third level high frequency components, second level high frequency components and first level high frequency components. This resulted in 48 features (2 moments×3 DWT signals×8 sensor signals).

2.2.3 Principal Component Analysis

The average scores of all the spatiotemporal features were used in a principal component analysis (PCA) to reduce the high dimensionality of the dataset, as there was a total of 88 features extracted for each hand. After the PCA a total of 15 principal components were retained (out of 29) which explained 94% of the total variation. Since the majority (220 observations, about 87%) of the mean TRS ratings were in the intervals between −2 and +1, it was decided that the principal components should be calibrated based on the values of the principal components that corresponded to the two tails of the distribution of the mean TRS value. The reasoning for this step was that this sampling would give the model a better ability to differentiate the three states and reduce the regression to mean effect. After taking into consideration the distribution of the mean TRS, PCA weights of the cases above 1 and below −2 were chosen to normalize the dataset; about 6.5% percent of each tail of the distribution (14 and 15 observations respectively out of total of 229 observations). The normalization was performed by subtracting the mean of the extreme cases for each of the 15 components and then dividing by the standard deviation.

It was further investigated what features contributed the most to the principal components. For each of the 88 features, a weighted average was calculated, based on the weight the features had for a specific component and the square root of the total variance explained from that component as:

$$FINAL SCORE = {\sum }_{i = 0}^{5} \vert weight\_i \vert * \sqrt{Variance explained\_i}$$

The intention was to investigate which features that contribute to the five most important components. That is because these explain about 68% of the variation. That procedure yielded a single value to represent the gravity of each feature.

2.2.4 Predictive Modelling—Computerized Assessment of MOTOR states

The calibrated principal components were used as predictors to supervised machine learning methods (Kubota et al. (2016)), to map the sensor data to the mean TRS ratings from the three specialists. To test the prediction performance of the model a 10-fold and a leave-one-individual out cross validation method were used. In the first case, the model was randomly split into 10 parts and at each step, 9 parts were used as the model training set and 1 part as the model testing set. The overall correlation between predicted and actual values together with prediction errors values were acquired. Only data points from the PD participants' data were used in the model building. At the leave-one-individual out procedure, all observations from one individual were left out of the training set, and those observations were used as the testing set. This produced 19 different prediction sets based on the number of the individuals. Again, correlation and error values were calculated.

SVM Model

A support vector machine model (SVM) was trained for regression. A bidirectional elimination method was used to select the components of the model. Principal components were included or excluded as predictors based on the performance of the SVM model on the testing set (error and correlation), in a 10-fold cross validation process. The 1st, 2nd, 3rd, 4th, 5th and 7th PCs were used as predictors in the final model, with the 1st PC modelled as a 3rd degree polynomial and the rest linearly added. That was because a visual inspection showed that there was a non-linear relationship between the first principal component and the TRS rating. Since it was the one that contained the most information a non-linear transformation was decided for that component, something that improved the predictive performance of the model in the cross-validation setting. Another important role in the selection of predictors, was the complexity of the final model, as simpler models, e.g. low number of predictors, are preferred.

The SVM model used a radial basis function and the parameters of the function were optimized, based on grid search of the parameter space. The optimized model had gamma value of 0.125, cost value of 8, epsilon value of 0.1 and 197 support vectors. The optimized parameters were the ones that reduced the mean squared error in a cross validation setting.

Decision Tree

A decision tree was trained for regression (ANOVA method). The model used the information gain criterion to choose between different splitting functions. The complexity parameter of the model was used to select the optimal tree size. The one that produced the lowest error in a cross validation setting was kept, a value of 0.05. All PC's were used in the model and the most important ones were decided from the two parameters described above. If introducing a new predictor would give gain in information, then this PC was built into the decision tree. The final model had 25 nodes, with 13 total PC's used in the tree.

Random Forest

In addition to decision trees, the random forest algorithm for regression was examined, building numerus decision trees. Then the values of the trees for a specific prediction are averaged. The number of trees used for this procedure was 500 and the number of variables for the split at each node was 3.

Linear Regression

For the linear model, the predictor selection was done with a backwards elimination method. All PC's were used as predictors in the initial model and at each step one predictor was removed, the one that had the highest p-value. The procedure stopped when all predictors in the model were significant, with a p-value threshold of 0.10. The 1st, 2nd, 3rd, 4th, 5th and 10th PCs were used as predictors in the final model, all linearly added in a model that had 220 degrees of freedom. Non-linear transformations of the PC's were examined but were deemed not significant.

2.2.5 Test Retest Reliability

Intraclass correlation (ICC) was used to assess the repeatability of the results of the TRIS. The baseline observations (before receiving any medication) were compared to the observations at the moment they received the dose (0 levodopa concentration). It was assumed that state of the participants between those observations was unchanged, not accounting for a placebo effect.

2.2.6 Sensitivity to Levodopa Treatment

The sensitivity to levodopa treatment was calculated based on the effect sizes of between occasions' changes. Sensitivity in this context means the ability of a measurement to detect a treatment effect. To estimate sensitivity, ANOVA models were fitted for each time point after the first test. The time points examined were: between 1st (baseline) and 2nd test, between 1st and 3rd test, . . . , between 1st and 10th test. The effects sizes acquired (η2-values) from the ANOVA models were used to calculate the sensitivity. The equation used was:

$${η}^{2} = \frac{Sum of Squares Treatmen}{Sum of squares Total}$$

The η²value (between 0 and 1) is the treatment effect size with the index having the highest value “defined as the most sensitive to treatment response” (Goetz et al. 2013).

2.2.7 Classification of PwP and Healthy Individuals

Machine learning models were also evaluated, regarding the classification ability between tests performed by healthy controls and PwP. The same models that were used for regression (SVM, decision tree, Random Forest, Linear classifier) were re-trained as binary classifiers for test occasions, using different components as predictors. 10-fold cross validation was used to test and validate the models performance against the testing set. The only result of interest was how many examples of the dataset the models could classify correctly.

3. Results:

Feature Importance

The 10 features that had the highest weights in the first 5 PC are presented in table 2. The mean of the rotation on the Z axis was the most important one, with the mean of rotation on the X-axis being the 4th most important. Two of the ApEn features are amongst the most important ones (ranked 3rd and 9th).

TABLE 2

| The 10 features that contribute the most on the construction of | |
|---|---|
| the first 5 principal components | |
| mrotZ | Mean rotation on the Z axis |
| staccY | Standard deviation of acceleration on the Y axis |
| aecomRot | ApEn of the magnitude of rotation |
| mrotX | Mean rotation on the X axis |
| stdcd2rotY | Standard deviation of DWT coefficients of rotation |
| | on the Y axis in the frequency range 12.8-25.6 Hz |
| strotZ | Standard deviation of rotation on the Z axis |
| stddwtaccZ | Standard deviation of combined DWT coefficients |
| | of acceleration on the Z axis |
| aerotX | ApEn of the rotation on the X axis |
| stddwtrotZ | Standard deviation of combined DWT coefficients |
| | of rotation on the Z axis |
| stdcd2rotZ | Standard deviation of DWT coefficients of rotation |
| | on the Z axis in the frequency range 12.8-25.6 Hz |

Further analysis in the leave-one-individual-out setting, when excluding 4 participants that showed no response to treatment (subjects 13, 14, 15 and 19 in FIG. 12), showed that the correlation and the error values for the SVM model are better than the linear regression model (correlation coefficient of 0.6 compared to 0.58 and RMSE of 0.91 compared to 0.94). Moreover, in the 10-fold CV (cross-validation) the SVM is clearly superior to the linear regression model.

Taking all that into consideration, it was decided to further examine the SVM model performance since, based at the performance in the 10-fold CV, it should be able to generalize better for future predictions. In that setting, the predicted SBLRI from the SVM was strongly correlated to mean TRS with the Pearson correlation coefficient being 0.82. The mean root square prediction error was 0.73 with a 95% confidence interval of ±1.67. In FIG. 11, a visual inspection of the model performance in the unseen data is presented. In the top plot, the residual vs. the fitted values are shown, with no apparent patterns. In the bottom plot the fitted vs. the actual values are shown, together with a line that goes through the origin. In that plot it is apparent that the values at the end tails of the TRS scale are over predicted for low values and under predicted for high values.

Leave One-Individual-Our Cross Validation

FIG. 12 shows the leave-one-individual out cross validation predictions of the SVM model, together with the individual levodopa concentrations are shown. The straight lines represent the TRS rating, the dashed lines the TRIS (left axis) and the dotted lines the levodopa concentration (right axis).

In FIG. 12, the leave-one-individual out cross validation predictions of the model, together with the individual levodopa concentrations are shown. The lines that join the diamond-shaped data points represents the mean TRS ratings, and the lines that join the triangular shaped data points the TRIS, for examples that the SVM has not seen before. Overall the TRIS has good correlation to the TRS and good response to levodopa concentration, in some cases better than the TRS (Participants 12, 13).

Sensitivity

FIG. 13 shows the sensitivity of TRS and TRIS based on the SVM model fit. The straight line represents the sensitivity of TRS rating and the dotted line the sensitivity of TRIS to levodopa response. Both show good sensitivity to levodopa, with peak values at 0.37 for the TRS and 0.33 for the TRIS. The TRIS sensitivity follows the same trend as the TRS sensitivity, even though overall it has lower values.

Test-Retest Reliability

To calculate the ICC correlation of the two baseline measurements of the states of the participants with PD, only 17 of the participants qualified. Those had an observation before they received the dose, and one observation at the moment they received the dose. The same levodopa concentration at these occasions is likely to give the same video ratings on the TRS scale and the same predictions for the SLTRI, barring any placebo effect. The results show very high ICC correlation in both scales, 0.89 in TRS scale (CI: 0.73, 0.96) and 0.83 in the TRIS predictions (CI: 0.6, 0.94), thus providing good test-retest repeatability. Two participants were removed from that analysis (7 and 15), since they had missing values at the first baseline observation.

Correlation to Other Clinical Ratings

In table 3, the TRIS correlation to other items of the UPDRS scale is shown, in comparison to the correlation of the TRS to the same items. The sensor index has high correlation to the gait, even though the participants were sitting, and the lowest correlation to the hand movements, even though these are the tests they performed. These ITEMS are highlighted in the table.

TABLE 3
Correlation of TRS and TRIS to the other items of the UPDRS
scale and dyskinesia. In the table two ITEMS are highlighted, the
rapid alternative hand movements (ITEM 25) and the gait (ITEM 29).
ITEM 25 has the lowest correlation, and ITEM 29 has the highest.

| | TRS | TRIS |
|---|---|---|
| ITEM 23 - finger tapping | −0.59 | −0.53 |
| ITEM 25 - rapid alternative movement | −0.46 | −0.44 |
| of hands | | |
| ITEM 27 - arising from chair | −0.59 | −0.56 |
| ITEM 29 - gait | −0.76 | −0.70 |
| ITEM 31 - bradykinesia | −0.86 | −0.81 |
| Dyskinesia | 0.71 | 0.67 |

Classification Models Based on the Principal Components Values

In was further investigated if classification algorithms can be trained with the ability to distinguish between tests performed by PwP and tests performed by healthy controls. In table 4, the performance of multiple classification algorithms is shown. Again the SVM model is the one with the best performance, followed by the linear regression model. In a 10-fold CV, the algorithms have the ability to correctly classify about 90% of the tests.

TABLE 4
Classification accuracy of the machine learning algorithms in a
validation dataset. The support vector machine model has the
best classification power.

| | Classification |
|---|---|
| | accuracy |
| Support vector | 0.89 |
| machines | |
| Linear regression | 0.80 |
| Decision tree | 0.73 |
| Random forest | 0.78 |

In this study the feasibility of building a sensor based treatment response index using data from wearable wrist sensors was examined. It was the intention to set up the basis of a practical method that people suffering with PD can reasonably use in their every-day life to obtain objective measurements. The data from the performed test (20 seconds wrist rotation test) were examined through time series spatiotemporal analysis.

Eighty-eight features were extracted from the sensors and then mapped to the TRS scale through machine earning algorithms. When examining which features were the most important, it could be asserted that the movement that contributed the most to the construction of the TRIS was the vertical movement of the participants' hands. It is imperative to notice though, that movements in all directions where amongst the most important. Furthermore, features from the whole extraction process contribute to the final model as features concerting accelerometry, rotation, DWT and ApEn all appear in the 10 most important ones in Table 2.

The convergence validity, test-retest reliability and the high sensitivity to levodopa treatment (similar to the TRS sensitivity), demonstrate that the TRIS had good clinimetric properties for measuring motor state in a scale from off to dyskinesia.

The results presented herein are an indication that the use of hand rotation tests can provide sufficient symptom information to map the state of the PwP into treatment response scales, deriving response scale values from objective measurements. An instrumented test, such as the one proposed, might be preferable to passive measurements. Passive measurements tend be are less precise and need repeated measurements over several days. That could cause discomfort since a proper off is uncomfortable to obtain, something necessary every morning for the measurement period (5-7 days).

The listing or discussion of an apparently prior-published document in this specification should not necessarily be taken as an acknowledgement that the document is part of the state of the art or common general knowledge.

### REFERENCES

Maetzler, W., Klucken, J. and Horne, M. (2016), A clinical view on the development of technology-based tools in managing Parkinson's disease. MovDisord.. doi:10.1002/mds.26673

Espay, A. J., Bonato, P., Nahab, F. B., Maetzler, W., Dean, J. M., Klucken, J., Eskofier, B. M., Merola, A., Horak, F., Lang, A. E., Reilmann, R., Giuffrida, J., Nieuwboer, A., Home, M., Little, M. A., Litvan, I., Simuni, T., Dorsey, E. R., Burack, M. A., Kubota, K., Kamondi, A., Godinho, C., Daneault, J.-F., Mitsi, G., Krinke, L., Hausdorff, J. M., Bloem, B. R., Papapetropoulos, S. and on behalf of the Movement Disorders Society Task Force on Technology (2016), Technology in Parkinson's disease: Challenges and opportunities. MovDisord.. doi:10.1002/mds.26642

Lopane, G., Mellone, S., Chiari, L., Cortelli, P., Calandra-Buonaura, G., &Contin, M. (2015). Dyskinesia detection and monitoring by a single sensor in PwP with Parkinson's disease. Movement Disorders, 30(9), 1267-1271.

Memedi M, Khan T, Grenholm P, Nyholm D, Westin J. Automatic and objective assessment of alternating tapping performance in Parkinson's disease. Sensors. 2013 Dec. 9; 13(12):16965-84.

Memedi, Mevludin; Aghanavesi, Somayeh; Westin, Jerker. Objective quantification of Parkinson's disease upper limb motor timing variability using spirography. 37th Annual International Conference of the IEEE Engineering in Medicine and Biology Society, 25-29 Aug. 2015, Milan, Italy

Stamatakis, Julien, et al. Finger tapping clinimetric score prediction in Parkinson's disease using low-cost accelerometers. Computational intelligence and neuroscience, 2013, 2013: 1.

Patel, Shyamal, et al. Monitoring motor fluctuations in patients with Parkinson's disease using wearable sensors. IEEE transactions on information technology in biomedicine, 2009, 13.6: 864-873.

Jane, Y. Nancy; Nehemiah, H. Khanna; Arputharaj, Kannan. A Q-backpropagated time delay neural network for diagnosing severity of gait disturbances in Parkinson's disease. Journal of biomedical informatics, 2016, 60: 169-176.

van Gilst, Merel M.; Bloem, Bastiaan R.; Overeem, Sebastiaan. Prospective assessment of subjective sleep benefit in Parkinson's disease. BMC neurology, 2015, 15.1: 1.

Griffiths, Robert I., et al. Automated assessment of bradykinesia and dyskinesia in Parkinson's disease. Journal of Parkinson's disease, 2012, 2.1: 47-55.

Tsipouras, Markos G., et al. “An automated methodology for levodopa-induced dyskinesia: assessment based on gyroscope and accelerometer signals.” artificial intelligence in Medicine 55.2 (2012): 127-135.

1. A system comprising:
a prescription-determination-module, configured to:
receive patient-model-data, wherein the patient-model-data comprises one or more dose-effect-parameters that represent how a patient is expected to react to a dose of medicament over time;
apply a plurality of different dosage-parameters to the patient-model-data, in order to determine a plurality of dosage-effect-data;
apply one or more selection-criteria to the plurality of dosage-effect-data in order to determine one or more of the associated dosage-parameters as selected-dosage-parameters; and
provide an output-signal based on the selected-dosage-parameters.

2. The system of claim 1, wherein the plurality of different dosage-parameters comprises a plurality of different dosage-intervals between discrete doses of a medicament.

3. The system of claim 1, wherein the plurality of different dosage-parameters comprises a plurality of different dosage-amounts for discrete doses of a medicament.

4. The system of claim 1, wherein the selection-criterion comprises an area-criterion, which is based on:
(i) a length of time that the dosage-effect-data spends outside an acceptable range for the dosage-effect-data, and
(ii) the extent to which the dosage-effect-data is outside the acceptable range.

5. (canceled)

6. The system of claim 1, wherein the medicament comprises levodopa and/or carbidopa; alternatively wherein the medicament comprises a medicament selected from levodopa/benserazide.

7-9. (canceled)

10. The system of claim 1, wherein the patient-model-data comprises one or more dose-effect-parameters that represent how a patient with Parkinson's Disease is expected to react to a dose of levodopa over time.

11. The system of claim 1, wherein the prescription-determination-module is configured to:
apply a first-dosage-parameter to the patient-model-data, in order to determine first-dosage-effect-data;
apply further-dosage-parameters, that are different to the first-dosage-parameter, to the patient-model-data in order to determine further-dosage-effect-data, until an end-criterion is satisfied.

12. (canceled)

13. The system of claim 1, wherein the system further comprises:
a model-builder, configured to:
receive a symptom-score that is associated with the patient whilst they performed an exercise, and an associated exercise-time, wherein the exercise-time is representative of a time that the patient performed the exercise,
receive a historic-dosing-schedule that includes at least one dose-taken-time, wherein the dose-taken-time is representative of a time that the patient took a dose of medicament; and
process the symptom-score, the associated exercise-time and the historic-dosing-schedule in order to determine the patient-model-data for the prescription-determination-module.

14. The system of claim 13, wherein the system further comprises:
a score-determination-module, configured to:
receive sensed-motion-signals that are representative of the patient's movement whilst they are performing the exercise; and
process the sensed-motion-signals in order to determine the symptom-score for the model-builder.

15-17. (canceled)

18. A method comprising:
applying a plurality of different dosage-parameters to patient-model-data, in order to determine a plurality of dosage-effect-data, wherein the patient-model-data comprises one or more dose-effect-parameters that represent how a patient is expected to react to a dose of medicament over time;
applying one or more selection-criteria to the plurality of dosage-effect-data in order to determine one or more of the associated dosage-parameters as selected-dosage-parameters; and
providing an output-signal based on the selected-dosage-parameters.

19-22. (canceled)

23. The method of anyone of claim 18, wherein the medicament comprises levodopa and/or carbidopa; alternatively wherein the medicament comprises a medicament selected from levodopa/benserazide.

24-26. (canceled)

27. The method of claim 18, wherein the patient-model-data comprises one or more dose-effect-parameters that represent how a patient with Parkinson's Disease is expected to react to a dose of levodopa over time.

28. The method of claim 18, further comprising:
applying a first-dosage-parameter to the patient-model-data, in order to determine first-dosage-effect-data;
applying further-dosage-parameters, that are different to the first-dosage-parameter, to the patient-model-data in order to determine further-dosage-effect-data, until an end-criterion is satisfied.

29. (canceled)

30. The method of claim 18, further comprising:
processing a symptom-score, an associated exercise-time and a historic-dosing-schedule in order to determine the patient-model-data for the prescription-determination-module, wherein:
the symptom-score is associated with the patient whilst they performed an exercise, and the associated exercise-time is representative of a time that the patient performed the exercise, and
the historic-dosing-schedule includes at least one dose-taken-time, wherein the dose-taken-time is representative of a time that the patient took a dose of medicament.

31. The method of claim 30, further comprising:
processing sensed-motion-signals in order to determine the symptom-score for the model-builder, wherein:
the sensed-motion-signals are representative of the patient's movement whilst they are performing the exercise.

32-36. (canceled)

37. A method of determining a personalised dosage regime for a patient for the treatment of a disease or disorder, the method comprising the steps described in claim 18.

38. The method of claim 37, wherein the disease or disorder is characterised by the modulation of musculo-skeletal movement.

39. The method of claim 38, wherein the disease or disorder is selected from primary or idiopathic Parkinsonism, Secondary Parkinsonism, hereditary Parkinsonism, Parkinson plus syndromes, Hallevorden-Spatz Disease, progressive supranuclearophthalmoplegia, striatonigral degeneration, dystonia, spasmodic torticollis, blepharospasm, essential tremor, unspecified tremors, myoclonus, chorea, athetosis, dyskinesia, tardive dyskenisia, tic disorders, Tourette's syndrome, stereotypic movement disorder, paroxysmal nocturnal limb movement, restless leg syndrome, stiff-person syndrome, fasciculation, epilepsy, seizures or ADHD.

40. The method of claim 37, wherein the medicament comprises:
levodopa and/or carbidopa or levodopa and/or benzaride;
dopamine agonists selected from aripiprazole, phencyclidine, quinpirole, salvinorin A, apomorphine, bromocriptine, cabergoline, ciladopa, dihydrexidine, dinapsoline, doxanthrine, epicriptine, lisuride, pergolide, piribedil, pramipexole, propylnorapomorphine, quinagolide, ropinirole, rotigotine, roxindole, sumanirole, fenoldopam, amphetamine, dextroamphetamine, bupropion, lisdexamfetamine, methylphenidate or dexmethylphenidate;
MOA-B inhibitors selected from isocarboxazid, nialamide, phenelzine, hydracarbazine, tranylcypromine, bifemelane, moclobemide, pirlindole, toloxatone, rasagiline or selegiline;
anti-epileptic and anticonvulsant selected from acetazolamide, carbamazepine, clobazam, clonazepam, eslicarbazepine acetate, ethosuximide, gabapentin, lacosamide, lamotrigine, levetiracetam, nitrazepam, oxcarbazepine, perampanel, piracetam, phenobarbital, phenytoin, pregabalin, primidone, retigabine, rufinamide, sodium valproate, stiripentol, tiagabine, topiramate, vigabatrin, zonisamide;
ADHD treatments selected from methylphenidate, dexamfetamine, lisdexamfetamine, atomoxetine or guanfacine; and
beta blockers selected from atenolol, bisoprolol, carvedilol, metoprolol, nebivolol, propranolol.

41. The method of claim 40 wherein the medicament comprises levodopa and/or carbidopa and the disease or disorder is selected from primary or idiopathic Parkinsonism, Secondary Parkinsonism, hereditary Parkinsonism, Parkinson plus syndromes.

42-44. (canceled)
